(19) United States
(12) Patent Application Publication
(10) Pub. No.: US 2003/0039957 A1
(43) Pub. Date: Feb. 27, 2003

(54) Functional protein expression for rapid cell-free phenotyping

(76) Inventors: Laurence McCarthy, Great Falls, VA (US); Lilly Kong, Covina, CA (US); Tang Shao, Rosemead, CA (US); Xin Su, Irvine, CA (US)

Correspondence Address: Ivor R. Elrifi, Ph.D., Mintz, Levin, Cohn, Ferris,, Glovsky and Popeo, P.C., One Financial Center, Boston, MA, 02111, US

(21) Appl. No.: 09/996,187

(22) Filed: Nov. 27, 2001

Related U.S. Application Data
(60) Provisional application No. 60/253,150, filed on Nov. 27, 2000.
(60) Provisional application No. 60/297,686, filed on Jun. 12, 2001.
(60) Provisional application No. 60/304,533, filed on Jul. 9, 2001.

(51) Int. Cl. C12Q 1/70; C12Q 1/68; G01N 33/53; G01N 33/554; G01N 33/569

(52) U.S. Cl. 435/5; 435/6; 435/7.1; 435/7.32

(57) ABSTRACT

Disclosed herein are methods for assaying the phenotype of a bioactive molecule in the presence and absence of compounds that are known inhibitors of the phenotypable activity of the bioactive molecule. Also disclosed are methods for discovering compounds that can inhibit the phenotypable activity of a bioactive molecule. The methods and assays of the present invention are useful in developing and monitoring a chemotherapy regimen for a patient, to detect or prevent the emergence of a drug resistant phenotype.

### RELATED APPLICATIONS

This application claims priority from U.S. provisional patents serial No. 60/253,150 filed Nov. 27, 2000, and serial No. 60/297,686 filed Jul. 12, 2001, incorporated herein by reference.

### FIELD OF THE INVENTION

The invention provides methods and compositions for detecting the phenotype of a bioactive molecule assays. More specifically, the invention provides methods and compositions for determining the suitability of one or more candidate compounds prior to or during the course of chemotherapy or anti-infective therapy, for their capacity to inhibit the bioactive molecules of micro-organisms, and cancers, and as an assay for expression in transgene therapy. Also provides are phenotypic assays for drug discovery.

### BACKGROUND OF THE INVENTION

It is generally known that microorganisms become resistant to drugs through evolution. Resistance to an anti-infective agent develops in microorganisms during the course of patient anti-infective therapy. Through mutational events at the molecular level, microorganisms modify the molecular structures of their proteins, most commonly enzymes that regulate growth or metabolism. Mutations are normal, and occur in the absence of anti-infective therapy, but mutations in proteins that are targets for anti-viral, anti-bacterial, and anti-fungal therapeutic agents can modify the affinities between the target and the agent, or prevent interaction or access to the target's active sites, thereby nullifying the agent's ability to deliver a therapeutic effect and destroy the microorganism. Drug therapy exerts a selection pressure on the microorganisms that selects for mutations that allow the microorganism to survive, resulting in re-infection of the patient with microbe displaying a new drug-resistant phenotype.

Drug resistance is now recognized as a common therapeutic complication in patient treatments with essentially all infective drugs. For example, penicillin, methicillin, and vancomycin resistance is often seen in anti-bacterial therapy and anti-retroviral agent resistance is commonly reported in anti-HIV therapies. Drug resistance can only be measured by limited methods for certain diseases, and HIV infection provides a well-studied example. For HIV infections, a viral load test (such as PCR, bDNA, and NASBA) can be used to determine viral replication levels in a patient. When a patient has a substantial increase in viral load while undergoing anti-retroviral drug therapy. This increase typically indicates the development of drug resistance. However, viral load tests do not assess directly the susceptibility of the virus to anti-viral compounds. Therefore, while load testing can be used to identify a patient whose virus may have developed resistance, this method cannot be used to determine the most effective drug for patient therapy. A method is needed for the evaluation and monitoring of a chemotherapeutic regimen at the onset and during the course of patient therapy.

Currently, the most common methods employed to measure resistance of HIV and other viral and bacterial infections to anti-infective agents are genotypic and phenotypic testing methods. Genotypic tests look for the presence of specific mutations that are known to cause resistance to certain drugs. These genotypic test methods are very time-intensive, requiring one to two weeks to generate conclusive test results, and suffer from further disadvantages. It can be difficult to translate mutational analysis data into meaningful clinical information useful in patient therapy, in cases, for example, where the mutation is novel or not well characterized. In fact, while HIV genotypic testing is widely used in clinical laboratories, this type of assay is not as well established for other diseases. Computer-assisted mutational interpretation programs used by scientists and clinicians do not yet share standard analytical algorithms, and keeping these algorithms current with the newest reported mutations in the scientific literature is difficult.

Phenotypic testing methods measure the actual susceptibility of the microbes to specific drugs. Traditional phenotypic assays require the ability to grow the disease-causing microbe in culture. Measuring the ability of drugs to inhibit bacterial growth has been a routine laboratory procedure for many years. The ability to culture the disease-causing microorganism from a patient specimen provides a first method to identify the microorganism and elect a therapeutic regimen. These assays also provide reliable in vitro methods of evaluating drug resistance or susceptibility to an anti-infective agent during the course of therapy, and thus can be used to monitor for the emergence or potential for drug resistance.

However, for viruses or cancers and certain fungi and bacteria, the methods of phenotypic analysis are both expensive and time-intensive, taking many weeks or months to complete. This disadvantage has hindered routine drug resistance analysis for viruses, such as CMV or HSV. Moreover, phenotypic testing cannot be applied to unculturable viruses, such as HCV. For HIV, a recombination phenotypic assay has been developed by inserting the amplified key components of patient-obtained HIV genetic material into engineered reference vectors of HIV in order to shorten this process. See, Petropoulos et al., Antimicrobial Agents and Chemotherapy, 44: 920-928 (2000) and Hertogs et al., Antimicrobial Agents and Chemotherapy, 42: 269-276 (1998), both incorporated herein by reference. While viral cultivation and propagation time has been reduced, this method still takes two to four weeks to produce the test results. In addition, the assay is labor intensive and tedious, requiring molecular construction of the vectors, cell culture and transfection, viral particle collection, and infection.

Thus, a need remains in the art for a more cost-effective and rapid phenotypic assay for measuring drug resistance in various diseases.

### SUMMARY OF THE INVENTION

The present invention provides phenotypic testing assays and methods for evaluating the suitability of a chemotherapeutic regimen for a patient afflicted with a disease state. Embodiments of the invention have applications in many disease states resulting from, for example, viral infections, bacterial infections, fungal infections, autoimmune disorders, genetic disorders, and cancers.

In one embodiment, the present invention is a diagnostic assay comprising reagents for extracting and purifying nucleic acid from an individual afflicted with a disease state, reagents for amplifying a nucleic acid sequence encoding one or more bioactive molecules expressed in the individual where the bioactive molecule is associated with the disease state, reagents for cell-free transcription of the amplified nucleic acid sequence encoding the bioactive molecule for cell-free translation of the amplified nucleic acid transcripts encoding the bioactive molecule, and reagents for phenotypic characterization of the polypeptide resulting from translation of the bioactive molecule, wherein the phenotype provides data useful for rapid evaluation or prediction of the response of an individual to at least one therapy designed to ameliorate the disease state.

In another embodiment, the reagents for amplifying the nucleic acid sequence encoding the bioactive molecule are used for polymerase chain reaction amplification of the nucleic acid sequence, such as a plurality of nucleic acid primers. In yet another such embodiment, the nucleic acid primers are nested. In still another embodiment, the primers have sequences selected from the group consisting of: SEQ ID NO:1, SEQ ID NO:2, SEQ ID NO:3, and SEQ ID NO:4 (see, Table 1). In still another aspect of the invention, the amplification of nucleic acids encoding the bioactive molecule further comprises adding one or more secondary nucleic acid sequences to the nucleic acid sequence encoding the bioactive molecule during the amplification steps. In one embodiment, these sequences can regulate transcription of the amplified nucleic acid. In another embodiment, these sequences encode polypeptides that facilitate purification of the bioactive molecule, for example, purification of the bioactive molecule by metal chelate chromatography, affinity chromatography, size exclusion chromatography, anion exchange chromatography, and cation exchange chromatography. In one embodiment, the purified bioactive molecules are studied for changes in their phenotype by, for example, changes assessing the bioactivity of a viral polymerase or a domain thereof, and its ability to catalyze DNA polymerization a nucleotide incorporation assay in the presence of one or more antiviral agents across a concentration range. Assays and methods useful to the present invention for determining enzyme structure and function, as well as target/ligand binding and dissociation kinetics include radioligand binding assays, protein co-immunoprecipitation, sandwiched ELISA, fluorescence resonance emission tomography (FRET), surface plasmon resonance (SPR), mass spectroscopy, nuclear magnetic resonance including 2-D NMR, and x-ray crystallography.

In one embodiment of the invention, the phenotypic assay comprises cell-free based assays and methods for transcription of the amplified nucleic acid sequence encoding the bioactive molecule, and cell-free translation of the nucleic acid transcripts thereby produced. In another embodiment, a coupled transcription/translation system, for example, a rabbit reticulocyte lysate system is employed. In a currently preferred embodiment, the coupled transcription/translation system does not require initial purification of the polymerase chain reaction amplification product. The present invention thus comprises assays and methods capable of generating sufficient quantities of the desired bioactive molecule for phenotypic characterization in a rapid manner, for example, 24 hours, 48 hours, or approximately one week.

In one embodiment, the present invention provides assays and methods comprising isolating nucleic acid from an individual infected with a virus, for example, the hepatitis B virus. In one aspect, a viral nucleic acid sequence encoding bioactive hepatitis B viral polymerase or a domain thereof is amplified by polymerase chain reaction, and from the nucleic acid isolated from the infected individual, the polymerase is transcribed and translated in a cell-free system. In another embodiment, the bioactivity of the viral polymerase or a domain thereof is characterized to determine the phenotype, which provides data useful for rapid evaluation or prediction of the response of the individual to at least one therapy designed to ameliorate the hepatitis B infection.

The assays and methods of the present invention have application in all areas of chemotherapy. In one aspect, the invention has applications in the field of anti-bacterial therapy, providing phenotype information to a physician about the bacteria that is causing the disease state in the patient, the information used in the selection and monitoring of an anti-bacterial chemotherapy regimen. In another aspect, the invention has applications in the field of anti-viral therapy, providing phenotype information to a physician about the virus that is causing the disease state in the patient, the information used in the selection and monitoring of an anti-viral chemotherapy regimen. In yet another aspect, the invention has applications in the field of anti-fungal therapy, providing phenotype information to a physician the fungus that is causing the disease state in the patient, the information used in the selection and monitoring of an anti-fungal chemotherapy regimen. In still another aspect, the invention has applications in the field of cancer therapy, providing phenotype information to a physician about the cancer that is causing the disease state in the patient, the information used in the selection and monitoring of an anti-cancer chemotherapy regimen. In another aspect, the invention has applications in the field of therapy directed against an autoimmune disorder, providing phenotype information to a physician about the autoimmune disorder that is causing the disease state in the patient, the information used in the selection and monitoring of an appropriate chemotherapy regimen. In yet another embodiment, the assay of the present invention is used to monitor the expression of proteins and protein markers during the course of gene replacement therapy, providing phenotypic information about the expressed gene product and its effects on metabolic pathways. In these embodiments, the present invention provides for phenotypic assays directed to a bioactive molecule implicated in a disease state, and methods of predicting and monitoring the bioactive molecule prior to or during a patient's chemotherapy regimen designed to ameliorate the disease state, and for evaluating the potential of newly developed drugs to treat the patient's affliction.

Methods and compositions embodied herein are envisioned for human and veterinary use. Veterinary use includes application to cows, horses, sheep, goats, pigs, dogs, cats, rabbits, and all rodents. The methods of the invention are also useful to agricultural workers and pet owners to combat infections contracted by exposure to livestock or pet animals.

In one aspect of the invention, phenotype data is obtained from an array of bioactive molecules. The phenotype data is recorded via a tangible medium, e.g., computer storage or hard copy versions. The data can be automatically input and stored by standard analog/digital (A/D) instrumentation that is commercially available. Also, the data can be recalled and reported or displayed as desired for best presenting the instant correlations of data. Accordingly, instrumentation and software suitable for use with the present methods are contemplated as within the scope of the present invention. Similarly, a database of phenotypic information for bioactive molecules is presented. The database uses standard relational database software, and can provide content through for example, CD ROM or the Internet.

A kit of the present invention comprises reagents for amplifying a nucleic acid sequence in a cell-free system, wherein the nucleic acid sequence comprises a bioactive molecule; reagents for expressing the bioactive molecule encoded by the nucleic acid sequence wherein the expressed bioactive molecule has a detectable phenotype, reagents for contacting the bioactive molecule with a compound; reagents for detecting the phenotype of the bioactive molecule in the presence or absence of the compound, and a first set of packaging materials comprising the reagents specified and a second set of packaging materials comprising the first set of packaging materials and user instructions. With particular regard to assay systems packaged in “kit” form, it is preferred that assay components be packaged in separate containers, with each container including a sufficient quantity of reagent for at least one assay to be conducted. As further described herein, one or more reagents may be labeled; alternatively, a labeling agent may be provided in the kit in its own container.

### BRIEF DESCRIPTION OF THE DRAWINGS

The following drawings illustrate the principles of the invention disclosed herein, are intended to be exemplary only, and should not be construed to limit the scope of the claims of the invention.

FIG. 1 illustrates an assay measuring the DNA dependent DNA polymerase activity of both mutant (HBV-m)and wild-type (HBV-WT) variants of the hepatitis B virus.

FIG. 2 illustrates an inhibition curve of the anti-viral compound lamivudine-TP, and its effects on wild-typeHBV polymerase activity over a concentration range of the drug.

FIG. 3 illustrates an inhibition curve of the anti-viral compound lamivudine-TP, and its effects on HBV polymerase activity over a concentration range of the drug as against the wild-type (HBV-WT) with a lamivudine sensitive phenotype and mutant HBV proteins with a lamivudine resistant phenotype (HBV-M, HM1, HM2, and HM5).

### DETAILED DESCRIPTION OF THE INVENTION

Definitions

As used herein in the specification and claims, the following words and phrases have the meanings as indicated.

“A viral disease state” refers to localized viral infections of tissues or systemic infection (viremia) in human and animal subjects. The bioactive molecules of viruses are detected and their phenotypes are observed. Examples of viral infections amenable to detection and monitoring by the invention disclosed herein comprise an adenovirus infection (such as infantile gastroenteritis, acute hemorrhagic cystitis, non-bacterial pneumonia, and viral conjunctivitis), a herpesvirus infection (such as herpes simplex type I and type II, varicella zoster (chicken pox), cytomegalovirus, and mononucleosis (Epstein-Barr virus)), a poxvirus infection (such as smallpox (variola major and variola minor), vaccinia virus, hantavirus and molluscum contagiosum), a picornavirus infection (such as rhinovirus (the common cold, also caused by coronavirus)) poliovirus (poliomyelitus)), an orthomyxovirus or paramyxovirus infection (such as influenza, and respiratory syncytial virus (RS)), parainfluenza virus (including such diseases as mumps), and rubeola (measles), a rhabdovirus infection (rabies), vesicular stomatitis (VSV), a togavirus infection such as encephalitis (EEE, WEE, and VEE), a flavivirus infection such as Dengue Fever, West Nile Fever, yellow fever, and encephalitis, bunyavirus and arenavirus, a togavirus infection such as rubella (German measles), a reovirus infection, a coronavirus infection, a hepatitis virus infection, a papovavirus infection such as papilloma virus, a retroviral infection such as HIV, HTLV-1, and HTLV-II.

“A bacterial disease state” refers to Gram positive and Gram negative bacterial infections in human and animal subjects. The bioactive molecules of bacteria are detected and their phenotypes are observed. Gram positive bacterial species are for example, genera including: Staphylococcus, such as S. epidermis and S. aureus; Micrococcus; Streptococcus, such as S. pyogenes, S. equis, S. zooepidemicus, S. equisimilis, S. pneumoniae and S. agalactiae; Corynebacterium, such as C. pyogenes and C. pseudotuberculosis; Erysipelothrix such as E. rhusiopathiae; Listeria, such as L. monocytogenes; Bacillus, such as B. anthracis; Clostridium, such as C. perfringens; and Mycobacterium, such as M. tuberculosis and M. leprae. Gram negative bacterial species are exemplified by, but not limited to genera including: Escherichia, such as E. coli 0157:H7; Salmonella, such as S. typhi and S. gallinarum; Shigella, such as S. dysenteriae; Vibrio, such as V. cholerae; Yersinia, such as Y. pestis and Y. enterocolitica; Proteus, such as P. mirabilis; Bordetella, such as B. bronchiseptica; Pseudomonas, such as P. aeruginosa; Klebsiella, such as K. pneumoniae; Pasteurella, such as P. multocida; Moraxella, such as M. bovis; Serratia, such as S. marcescens; Hemophilus, such as H. influenza; and Campylobacter species. Other species suitable for assays of the present invention include Enterococcus, Neisseria, Mycoplasma, Chlamidia, Francisella, Pasteurella, Brucella, and Enterobacteriaceae. Further examples of bacterial pathogenic species that are inhibited according to the invention are obtained by reference to standard taxonomic and descriptive works such as Bergey's Manual of Determinative Bacteriology, 9^{th} Ed., 1994, Wiiams and Wilkins, Baltimore, Md.

“A fungal disease state” refers to fungal infections in human and animal subjects. The bioactive molecules of fungi are detected and their phenotypes are observed. Examples of fungal genera are for example, Candida, such as C. albicans; Cryptococcus, such as C. neoformans; Malassezia (Pityrosporum); Histoplasma, such as H. capsulatum; Coccidioides, such as C. immitis; Hyphomyces, such as H. destruens; Blastomyces, such as B. dermatiditis; Aspergillus, such as A. fumigatus; Penicillium, such as P. marneffei; Pseudallescheria; Fusarium; Paecilomyces; Mucor/Rhizopus; and Pneumocystis, such as P. carinii. Subcutaneous fungi, such as species of Rhinosporidium and Sporothrix, and dermatophytes, such as Microsporum and Trichophyton species, are amenable to prevention and treatment by embodiments of the invention herein. Other disease casing fungi include Trichophyton, Microsporum; Epidermophyton; Basidiobolus; Conidiobolus; Rhizopus Cunninghamelia; Rhizomucor; Paracoccidioides; Pseudallescheria; Rhinosporidium; and Sporothrix.

“A protozoal disease state” refers to infection with one or more single-celled, usually microscopic, eukaryotic organisms, such as amoebas, ciliates, flagellates, and sporozoans, for example, Plasmodium, Trypanosoma or Cryptosporidium. The bioactive molecules of protozoa are detected and their phenotypes are observed.

“A cancer disease state” refers to any of various malignant neoplasms characterized by the proliferation of anaplastic cells that tend to invade surrounding tissue and metastasize to new body sites. For example, lung cancer, pancreatic cancer, colon cancer, ovarian cancer, cancers of the liver, leukemia, lymphoma, melanoma, thyroid follicular cancer, bladder carcinoma, glioma, myelodysplastic syndrome, breast cancer or prostate cancer. The bioactive molecules of diseased cells and their phenotype are observed.

“An autoimmune disease state” refers to an immune response by the body against one of its own tissues, cells, or molecules, wherein the immune response creates a pathological disease state. The bioactive molecules of a pathological immune response are detected and their phenotypes are observed. Examples of immune disorders comprise such disorders as systemic lupus erythematosus, (SLE), rheumatoid arthritis, Crohn's disease, asthma, DiGeorge syndrome, familial Mediterranean fever, immunodeficiency with Hyper-IgM, severe combined immunodeficiency, ulcerative colitis, Graves disease, autoimmune hepatitis, autoimmune thrombocytopenia, myesthenia gravis, sjogren's syndrome, and scleroderma.

“A genetic disease state” refers to a disease state resulting from the presence of a gene, the expression product of the gene being a bioactive molecule that causes or contributes to the disease state, or the absence of a gene where the expression product of the gene in a healthy individual is a bioactive molecule that ameliorates or prevents the disease state. The bioactive molecules of an expressed transgene are detected and their pheotypes are observed. An example of the former is cystic fibrosis, wherein the disease state is caused by mutations in the CFTR protein. An example of the latter is PKU, where the disease state is caused by the lack of an enzyme permitting the metabolism of phenylalanine. Examples of genetic disorders appropriate for screening with the present assays and methods include, for example Alzheimer disease, Amyotrophic lateral sclerosis, Angelman syndrome, Charcot-Marie-Tooth disease, Epilepsy, Essential tremor, Fragile X syndrome, Friedreich's ataxia, Huntington disease, Niemann-Pick disease, Parkinson disease, Prader-Willi syndrome, Rett syndrome, Spinocerebellar atrophy, Williams syndrome, Ellis-van Creveld syndrome, Marfan syndrome, Myotonic dystrophy, leukodystrophy, Atherosclerosis, Best disease, Gaucher disease, Glucose galactose malabsorption, Gyrate atrophy, Juvenile onset diabetes, Obesity, Paroxysmal nocturnal hemoglobinuria, Phenylketonuria, Refsum disease, and Tangier disease.

“Amplification reaction mixture” and “polymerase chain reaction mixture” refer to a combination of reagents that is suitable for carrying out a polymerase chain reaction. The reaction mixture typically consists of oligonucleotide primers, nucleotide triphosphates, and a DNA or RNA polymerase in a suitable buffer.

“Amplification conditions”, as used herein, refers to reaction conditions suitable for the amplification of the target nucleic acid sequence. The amplification conditions refer both to the amplification reaction mixture and to the temperature cycling conditions used during the reaction.

“Anti-microbial” activity of an agent or composition shall mean the ability to inhibit growth of one or more microorganisms. For example, the anti-microbial compositions described herein inhibit the growth of or kill bacterial, algal, fungal, protozoan, and viral genera and species thereof. It is well known to one of skill in the art of antibiotics development that an agent that causes inhibition of growth can also be lethal to the microorganism (bacteriocidal, for example in the case of a microorganism that is a bacterium).

“Bioactive molecule” means a nucleic acid, ribonucleic acid, polypeptide, glycopolypeptide, mucopolysaccharide, lipoprotein, lipopolysaccharide, carbohydrate, enzyme or co-enzyme, hormone, chemokine, lymphokine, or similar compound, that involves, regulates, or is the rate-limiting compound in a biosynthetic reaction or metabolic or reproductive process in a microorganism or tissue. Such bioactive molecules are common therapeutic drug targets, and include for example and without limitation, interferon, TNF, v-Ras, c-Ras, reverse transcriptase, g-coupled protein receptors (GPCR's), FcεR's, FcγR's, nicotinicoid receptors (nicotinic receptor, GABA_{A} and GABA_{C} receptors, glycine receptors, 5-HT₃ receptors and some glutamate activated anionic channels), ATP-gated channels (also referred to as the P2X purinoceptors), glutamate activated cationic channels (NMDA receptors, AMPA receptors, Kainate receptors, etc.), hemagglutinin (HA), receptor-tyrosine kinases (RTK's) such as EGF, PDGF, NGF and insulin receptor tyrosine kinases, SH2-domain proteins, PLC-γ, c-Ras-associated GTPase activating protein (RasGAP), phosphatidylinositol-3-kinase (PI-3K) and protein phosphatase 1C (PTP1C), as well as intracellular protein tyrosine kinases (PTK's), such as the Src family of tyrosine kinases, glutamate activated cationic channels (NMDA receptors, AMPA receptors, Kainate receptors, etc.), protein-tyrosine phosphatases Examples of receptor tyrosine phosphatases include: receptor tyrosine phosphatase rho, protein tyrosine phosphatase receptor J, receptor-type tyrosine phosphatase D30, protein tyrosine phosphatase receptor type C polypeptide associated protein, protein tyrosine phosphatase receptor-type T, receptor tyrosine phosphatase gamma, leukocyte-associated Ig-like receptor ID isoform, LAIR-1D, LAIR-1C, MAP kinases, neuraminadase (NA), proteases, polymerases, serine/threonine kinases, second messengers, transcription factors, and other such important metabolic building blocks or regulators. Virtually any bioactive molecule can be monitored with the present invention.

“Broad spectrum” anti-microbial activity means to ability to inhibit growth of organisms that are relatively unrelated. For example, ability of an agent to inhibit growth of both a Gram positive and a Gram negative bacterial species is considered a broad spectrum activity, as is the ability to inhibit growth of different microorganisms, such as a bacteria and a fungus.

“Hybridization” refers to the formation of a duplex structure by two single-stranded nucleic acids due to complementary base pairing. Hybridization can occur between fully (exactly) complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions”. Stable duplexes of substantially complementary sequences can be achieved under less stringent hybridization conditions. Those skilled in the art of nucleic acid technology can determine duplex stability empirically following the guidance provided by the art (see, e.g., Sambrook et al., Molecular Cloning—A Laboratory Manual, Cold Spring Harbor Laboratory, Cold Spring Harbor, N.Y., (1989), incorporated herein by reference).

“Nested” and “nested primers” means at least two nucleic acid oligonucleotide sequences where at least one first primer sequence (the internal sequence) comprises a part of the other primer (the external sequence), to constitute a nested primer set. Nested primer PCR generally involves a pair of nested primer sets, (for example an upstream nested primer set and a downstream nested primer set) and is used, for example but without limitation, to increase yields of the desired amplification target where there is little starting material to use as a template, or where the sample is contaminated with other nucleic acid material that can provide an undesirable false priming template (see, Sambrook et al., (1989) for a further description of nested primer design and use).

“Nucleic acid” shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine base. These terms refer only to the primary structure of the molecule. Thus, these terms include double- and single-stranded DNA, as well as double- and single-stranded RNA including tRNA. The terms “nucleic acid primer” and “oligonucleotide” refer to primers, probes, and oligomer fragments to be amplified or detected. There is no intended distinction in length between the terms “nucleic acid primer” and “oligonucleotide”, and these terms will be used interchangeably.

“Detecting the phenotype” means determining the physical properties of a bioactive molecule, for example a drug resistant phenotype, a drug sensitive phenotype, a change in the kinetics of the bioactive molecule or binding affinity for a particular ligand or therapeutic agent, a change in an epitope, catalytic site or other structural change to a bioactive molecule, loss or gain of function, and any such qualitative or quantitative experiment or diagnostic used to analyze these properties. The phenotype thus refers to observable physical or biochemical characteristics of a bioactive molecule or traits of an organism that expresses the bioactive molecule based on, for example, genetic and environmental influences.

“Primer” refers to an oligonucleotide capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer but typically ranges from 10 to 50 nucleotides. Short primer molecules generally require cooler temperatures to form sufficiently stable hybrid complexes with the template. A primer need not reflect the exact sequence of the template nucleic acid, but must be sufficiently complementary to hybridize with the template. Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning of the amplified product. The region of the primer, which is sufficiently complementary to the template to hybridize, is referred to herein as the hybridizing region.

An oligonucleotide primer or probe is “specific” for a target sequence if the number of mismatches present between the oligonucleotide and the target sequence is less than the number of mismatches present between the oligonucleotide and non-target sequences. Hybridization conditions between primers and template sequences for PCR can be chosen under which stable duplexes are formed only if the number of mismatches present is no more than the number of mismatches present between the oligonucleotide and the target sequence. Under such conditions, the target-specific oligonucleotide can form a stable duplex only with a target sequence. The use of target-specific primers under suitably stringent amplification conditions enables the specific amplification of those target sequences, which contain the target primer binding sites. Similarly, the use of target-specific probes under suitably stringent hybridization conditions enables the detection of a specific target sequence.

“Target region” and “target nucleic acid” refers to a region of a nucleic acid, which is to be amplified, detected, or otherwise analyzed. The sequence to which a primer or probe hybridizes can be referred to as a “target.”

“Thermostable DNA polymerase” refers to an enzyme that is relatively stable to heat and catalyzes the polymerization of nucleoside triphosphates to form primer extension products that are complementary to one of the nucleic acid strands of the target sequence. The enzyme initiates synthesis at the 3′ end of the primer and proceeds in the direction toward the 5′ end of the template until synthesis terminates. Purified thermostable DNA polymerases are commercially available from Perkin-Elmer, (Norwalk, Conn.).

An “upstream” primer refers to a primer whose extension product is a subsequence of the coding strand; a “downstream” primer refers to a primer whose extension product is a subsequence of the complementary non-coding strand. A primer used for reverse transcription, referred to as an “RT primer”, hybridizes to the coding strand and is thus a downstream primer.

Conventional techniques of molecular biology and nucleic acid chemistry, which are within the skill of the art, are fully explained in the literature. See, for example, Sambrook et al., 1989, Molecular Cloning—A Laboratory Manual, Cold Spring Harbor Laboratory, Cold Spring Harbor, N.Y.; Oligonucleotide Synthesis (M. J. Gait, ed., 1984); Nucleic Acid Hybridization (B. D. Hames and S. J. Higgins. eds., 1984); and a series, Methods in Enzymology (Academic Press, Inc.), all of which are incorporated herein by reference. All patents, patent applications, and publications mentioned herein, both supra and infra, are incorporated herein by reference.

### DETAILED DESCRIPTION OF THE DRAWINGS

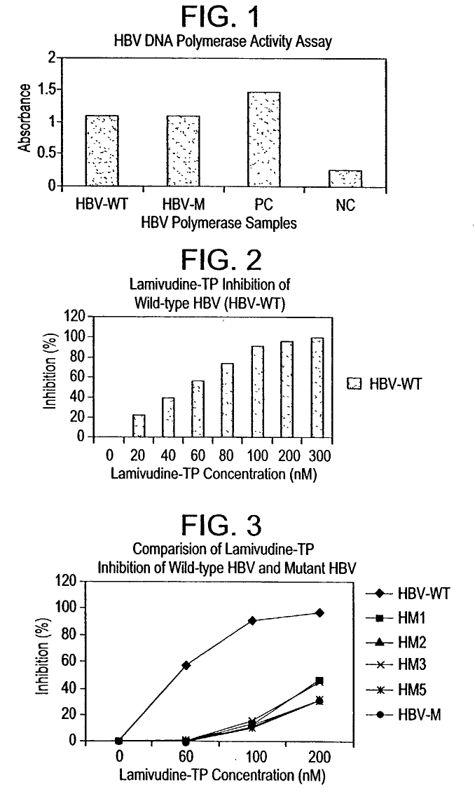

FIG. 1 illustrates an assay measuring the DNA dependent DNA polymerase activity of both mutant and wild-type variants of the hepatitis B virus (HBV). The DNA polymerase assay as shown provides a non-radioactive assay, which measures the ability of the enzyme to incorporate modified nucleotides into freshly synthesized DNA. The detection of synthesized DNA as a parameter for DNA polymerase activity follows a sandwich ELISA protocol. The absorbence the samples is directly correlated to the level of DNA polymerase activity in the sample. HBV-WT refers to the wild-type HBV polymerase. HBV-M refers to an HBV polymerase containing a type-I mutation (L528M and M552V), that is phenotypicaly associated with lamivudine resistance. PC and NC refer respectively to positive and negative controls (see, Example 1).

FIG. 2 illustrates an inhibition curve of the anti-viral compound lamivudine-TP, and its effects on wild-type HBV polymerase activity over a concentration range of the drug. Lamivudine-TP was added to the polymerase assay across a final concentration range of 0, 20, 40, 60, 80, 100, 200, and 300 nM. Inhibition of DNA polymerase activity (%) was plotted against drug concentration. The curve defines the enzymes sensitivity across the compounds range.

FIG. 3 illustrates an inhibition curve of the anti-viral agent lamivudine-TP, and its effects on HBV polymerase activity over a concentration range of the drug as against the wild-type HBV polymerase (HBV-WT), the type-I mutant HBV protein (HBV-M, HM2 and HM5), and the type-II mutants (HM1 and HM3, displaying M552I and also phenotypically associated with lamivudine resistance). Lamivudine-TP was added to the polymerase assay across a final concentration range of 0, 60, 100, and 200 nM. Inhibition of DNA polymerase activity (%) was plotted against drug concentration. Thus, a phenotype and a sensitive resistant phenotype for HBV polymerase to lamivudine is detected.

### DETAILED DESCRIPTION

The present invention provides phenotypic testing assays and methods for evaluating the suitability of a chemotherapeutic regimen for a patient afflicted with one or more disease states. The invention has applications in many types of disease states, but preferred diseases particularly suited to the assays and methods disclosed herein are viral infections, bacterial infections, fungal infections, autoimmune disorders, genetic disorders and cancers, wherein a bioactive molecule displaying phenotypable activity is implicated in, or known to be present in the disease state. Preferably, the bioactive molecule is a direct target for a chemotherapeutic agent. Thus, a direct correlation can be made between the molecule's phenotype and a agent's clinical efficacy. However, the invention also has application in assays where the bioactive molecule demonstrating a phenotype capable of detection is not the direct drug target, but instead lies downstream in a metabolic pathway from the drug target, i.e., in an enzyme cascade or cycle. It is desirable but not necessary that the phenotypable bioactive molecule be involved in a rate-limiting reaction, or be unique to the particular infective microorganism, or expressed in quantifiably different levels in disease tissues compared to healthy tissues as detectable by, for example, quantitative RT-PCR, so as to provide supplementary data to clinicians. PCR and similar amplification techniques are sensitive enough to amplify even low-level transcripts expressed weakly or transiently in a tissue such as a cancer tissue, or in slow replicating viruses or microorganisms.

A subject is diagnosed as having a microbial infection such as, a viral . . . or a cancer by inspection of a bodily tissue, e.g., epidermal and mucosal tissue, including such tissue present in surfaces of oral, buccal, anal, and vaginal cavities. Diagnosis of infection is made according to criteria known to one of skill in the medical arts, including but not limited to, areas of inflammation or unusual patches with respect to color, dryness, exfoliation, exudation, prurulence, streaks, or damage to integrity of surface. Conditions exemplary of those treated by the compositions and methods herein, such as abscess, meningitis, cutaneous anthrax, septic arthritis, emphysema, impetigo, cellulitis, pneumonia, sinus infection and tubercular disease are accompanied by elevated temperature. Diagnosis can be confirmed using standard ELISA-based kits, and by culture, and by traditional stains and microscopic examination of direct samples, or of organisms cultured from an inoculum from the subject. The preferred method of confirming diagnosis is isolation and identification of a disease-specific polynucleotide or polypeptide from an individual as described herein. Diagnosis often reveals the presence of one or more disease states in a patient, for example, patients that become severely immunocompromised because of underlying diseases such as leukemia or acquired immunodeficiency syndrome or patients who undergo cancer chemotherapy or organ transplantation, are particularly susceptible to opportunistic fungal infections. The invention is particularly suited to detecting multiple bioactive molecules from the etiological agent of one or more disease states in a single assay, for example, by using multiple primer sets in a single PCR amplification.

Amplification

The polymerase chain reaction (PCR) amplification process is well known in the art and described in U.S. Pat. Nos. 4,683,195; 4,683,202; and 4,965,188, incorporated herein by reference. Commercial vendors, such as Perkin Elmer (Norwalk, Conn.), market PCR reagents and publish PCR protocols. For ease of understanding, the advantages provided by the present invention, a summary of PCR is provided.

In each cycle of a PCR amplification, a double-stranded target sequence is denatured, primers are annealed to each strand of the denatured target, and the primers are extended by the action of a DNA polymerase. The process is repeated typically at least 7 and up to 35 times, but this will vary depending on the desired experimental conditions. The two primers anneal to opposite ends of the target nucleic acid sequence and in orientations such that the extension product of each primer is a complementary copy of the target sequence and, when separated from its complement, can hybridize to the other primer. Each cycle, if it were 100% efficient, would result in a doubling of the number of target sequences present.

Either DNA or RNA target sequences can be amplified by PCR. In the case of an RNA target, such as in the amplification of HBV nucleic acid as described herein, the first step consists of the synthesis of a DNA copy (cDNA) of the target sequence. The reverse transcription can be carried out as a separate step, or preferably in a combined reverse transcription-polymerase chain reaction (RT-PCR), a modification of the polymerase chain reaction for amplifying RNA. The RT-PCR amplification of RNA is well known in the art and described in U.S. Pat. Nos. 5,322,770 and 5,310,652; Myers and Gelfand, Biochemistry 30(31): 7661-7666 (1991); Young et al., J. Clin. Microbiol. 31(4): 882-886 (1993); and Young et al., J. Clin. Microbiol. 33(3): 654-657 (1995); each incorporated herein by reference.

Various sample preparation methods suitable for RT-PCR have been described in the literature. For example, techniques for extracting ribonucleic acids from biological samples are described in Rotbart et al., in PCR Technology (Erlich ed., Stockton Press, N.Y. (1989)) and Han et al., Biochemistry 2: 1617-1625 (1987), both incorporated herein by reference. The particular method used is not a critical part of the present invention. One of skill in the art can optimize reaction conditions for use with the known sample preparation methods. Due to the enormous amplification possible with the PCR process, low levels of DNA contamination from samples with high DNA levels, positive control templates, or from previous amplifications can result in PCR products, even in the absence of purposefully added template DNA. Laboratory equipment and techniques which will minimize cross contamination are discussed in Kwok and Higuchi, Nature, 339: 237-238 (1989), and Kwok and Orrego, in: Innis et al., eds., PCR Protocols: A Guide to Methods and Applications, Academic Press, Inc., San Diego, Calif. (1990), which are incorporated herein by reference. Enzymatic methods to reduce the problem of contamination of a PCR by the amplified nucleic acid from previous reactions are described in PCT patent publication No. U.S.91/05210, U.S. Pat. No. 5,418,149, and U.S. Pat. No. 5,035,996, each incorporated herein by reference.

Amplification reaction mixtures are typically assembled at room temperature, well below the temperature needed to insure primer hybridization specificity. Non-specific amplification may result because at room temperature the primers may bind non-specifically to other, only partially complementary nucleic acid sequences, and initiate the synthesis of undesired nucleic acid sequences. These newly synthesized, undesired sequences can compete with the desired target sequence during the amplification reaction and can significantly decrease the amplification efficiency of the desired sequence. Non-specific amplification can be reduced using a “hot-start” wherein primer extension is prevented until the temperature is raised sufficiently to provide the necessary hybridization specificity.

In one hot-start method, one or more reagents are withheld from the reaction mixture until the temperature is raised sufficiently to provide the necessary hybridization specificity. Hot-start methods which use a heat labile material, such as wax, to separate or sequester reaction components are described in U.S. Pat. No. 5,411,876 and Chou et al., Nucl. Acids Res., 20(7): 1717-1723 (1992), both incorporated herein by reference. In another hot-start method, a reversibly inactivated DNA polymerase is used which does not catalyze primer extension until activated by a high temperature incubation prior to, or as the first step of, the amplification. Non-specific amplification also can be reduced by enzymatically degrading extension products formed prior to the initial high-temperature step of the amplification, as described in U.S. Pat. No. 5,418,149, which is incorporated herein by reference.

Amplification of nucleic acids in the present invention can also be effectuated by amplification methods such as ligase chain reaction (LCR), transcription mediated amplification, (TMA), nucleic acid sequence based amplification (NASBA), ligation activated transcription (LAT), and strand displacement amplification (SDA). These techniques can provide bioactive molecules (nucleic acids) in micromolar concentration from femtomolar target template concentrations.

Ligase chain reaction (LCR), works by using two differently labeled halves of a sequence of interest which are covalently bonded by ligase in the presence of the contiguous sequence in a sample, forming a new target. LAT works from a single-stranded template with a single primer that is partially single-stranded and partially double-stranded. Amplification is initiated by ligating a cDNA to a promoter oligonucleotide and within a few hours, amplification is 10⁸ to 10⁹-fold. Nucleic acid amplification by strand displacement activation (SDA) utilizes a short primer containing a recognition site for HincII with a short overhang on the 5′ end which binds to target DNA. A DNA polymerase fills in the part of the primer opposite the overhang with sulfur-containing adenine analogs. Following amplification, HincII is added to cut the unmodified DNA strand. A DNA polymerase that lacks 5′ exonuclease activity enters at the site of the nick and begins to polymerize, displacing the initial primer strand downstream and building a new one which serves as more primer. SDA produces greater than 1-fold amplification in 2 hours at 37° C. Unlike PCR and LCR, SDA does not require instrumented temperature cycling. See, U.S. Pat. Nos. 6,312,908 and 6,316,200, incorporated herein by reference, for nucleic acid amplification methods. Although PCR is the preferred method of amplification of the invention, these other methods can also be used to amplify the target nucleic acid as described in the method of the invention.

Primers

Oligonucleotide primers can be prepared by any suitable method, including, for example, cloning and restriction of appropriate sequences and direct chemical synthesis by a method such as the phosphotriester method of Narang et al., 1979, Meth. Enzymol. 68: 90-99; the phosphodiester method of Brown et al., 1979, Meth. Enzyme. 68: 109-151; the diethylphosphoramidite method of Beaucage et al., 1981, Tetrahedron Lett. 22: 1859-1862; and the solid support method of U.S. Pat. No. 4,458,066, each incorporated herein by reference. Methods for synthesizing labeled oligonucleotides are described in Agrawal and Zamecnik, 1990, Nucl. Acids. Res. 18(18): 5419-5423; MacMillan and Verdine, 1990, J. Org. Chem. 55: 5931-5933; Pieles et al., 1989, Nucl. Acids. Res. 17(22): 8967-8978; Roget et al., 1989, Nucl. Acids. Res. 17(19): 7643-7651; and Tesler et al., 1989, J. Am. Chem. Soc. 111: 6966-6976, each incorporated herein by reference. A review of synthesis methods is provided 1990, Bioconjugate Chemistry 1(3): 165-187, incorporated herein by reference. Table 1 illustrates a nested primer set of the present invention, used to amplify the viral gene encoding HBV polymerase. One or more secondary nucleic acid sequences may be added to the nucleic acid sequence encoding the bioactive molecule by PCR during the amplification steps depending on the experimental strategy, for example, these secondary nucleic acid sequences include His tags, HA or FLAG epitopes or other immunological based purification motifs, GST, streptaviden or MBP proteins, nucleic acid sequences or other purification motifs. Methods of purification of recombinant proteins are well described, and such methods applicable to the invention include metal chelate chromatography, affinity chromatography, size exclusion chromatography, anion exchange chromatography, and cation exchange chromatography. These purification techniques can also be employed with such chromatography systems as a gas chromatograph, HPLC or FPLC. The secondary nucleic acid sequences may comprise sequences encoding regulatory elements that modulate transcription or translation of the gene in the amplified nucleic acid, for example but not limited to, by adding a promoter such as ADH, T7, RSV, or CMV promoter, or by adding a Kozak sequence, or stem-loop termination sequences. Other reporter genes or domains may be used to create fusion proteins with the polypeptide of interest, for example, a GFP fusion protein or β-galactosidase fusion protein. The invention also contemplates that multiple primer sets can be used to amplify one or more bioactive targets from a single reaction. The use of secondary nucleic acid sequences provides a particular advantage of the present invention where it is desirable that the nucleic acid sequences encoding the bioactive molecule are to be purified or cloned directly from a single PCR reaction that also generates the protein for the phenotypic assay.

TABLE 1

| | Primer | |
|---|---|---|
| SEQ ID NO: | Designation | Size/Type/Origin/Sequence |
| 1 | HB10 | 27 bases/single stranded linear DNA/artificial sequence/ |
| | | 5′-cctatagaccaccaaatgcccctatct-3′ |
| 2 | HB11 | 24 bases/single stranded linear DNA/artificial sequence/ |
| | | 5′-aggagatctctgacggaaggaaag-3′ |
| 3 | HB3 | 61 bases/single stranded linear DNA/artificial sequence/ |
| | | 5′-gaaattaatacgactcactataggagaaggagaaccatgcccctatcttatcaacacttcc-3′ |
| 4 | HB6 | 27 bases/single stranded linear DNA/artificial sequence/ |
| | | 5′-tttttacggtcgttgacattgctggga-3′ |

In vitro Transcription and Translation

Assays and methods of the present invention comprise expression systems for transcribing the amplified cDNA encoding the bioactive molecule, and for translating the RNA, into the bioactive molecule in a cell-free expression system. It is preferred that a coupled transcription/translation system is used that can use linear DNA, i.e., PCR-amplified DNA, as a starting material. Since the PCR-amplified nucleic acids are used directly as templates for protein expression, it eliminates plasmid-based cloning procedures for protein expression and cell culture (see, Li et al., Biochem. Cell Biol., 77: 119-126 (1999), Kim et al., Virus Gene, 19: 123-130 (1999), Qadri et al., J. Biol. Chem., 274: 31359-31365 (1999), Xiong et al., Hepatology, 28: 1669-1673 (1998), Seifer et al., J. Virol., 72: 2765-2776 (1998), Lee et al., Biochem. Biophys. Res. Comm., 223: 401-407 (1997), Landford et al., J. Virol., 69: 4431-4439 (1995), Tavis et al., Proc. Natl Acad. Sci. 90: 4107-4111 (1993), and U.S. Pat. Nos. 5,655,563; 5,552,302; 5,492,817; 5,324,637; 4,966,964, all incorporated herein by reference). Commercially available expression systems are the TNT® SP6 Coupled Reticulocyte Lysate System, TNT® T7 Coupled Reticulocyte Lysate System, TNT® T3 Coupled Reticulocyte Lysate System, TNT® T7/T3 Coupled Reticulocyte Lysate System, TNT® T7/SP6 Coupled Reticulocyte Lysate System, and the TNT® T7 Quick for PCR Coupled Reticulocyte Lysate System by Promega. The technical manuals of these assays are hereby incorporated by reference. The ability to amplify a target and incorporate secondary nucleic acid sequences into the amplicons such as the T7, T3 and SP6 promoters permits the expression of multiple polypeptides in a single cell-free reaction, such as an enzyme and a co-factor, or multiple subunit domains of an enzyme. Other expression systems are known to those skilled in the art, and are useful with the invention described herein. These other systems are considered to be within the scope of this invention. For example, an E. coli lysate system has also been used (Roche Molecular Biochemicals, Indianapolis, Ind.). Without being limited to theory, it is preferred that the coupled expression system use lysate from mammalian cells or eukaryotic cells so as to insure correct post-translational modification of the bioactive molecule, i.e., RNA processing or protein processing such as glycosylation. In a currently preferred embodiment, the translation or coupled transcription/translation system does not require initial purification of the polymerase chain reaction amplification product, and protein expression can proceed directly from the amplification step. Generally, about 1-500 pMols of nucleic acid is sufficient for the translation reaction, yielding approximately 0.1-100 μMols of protein. The expression system functions with all nucleic acids including synthetic nucleic acid sequences, which are considered to be within the scope of this invention.

Phenotype Assays

The phenotypes of the bioactive molecules are observed and detected by, for example, changes assessing the bioactivity of a viral polypeptide or a domain thereof, and its effects in a nucleotide incorporation assay in the presence and absence of one or more antiviral agents. One such assay is described in Example 1, and measures the ability of a viral polymerase to catalyze the incorporation of fluorescent-labeled nucleotides into nascent DNA in the presence of a concentration range of an anti-viral agent. Another assay capable of detecting a phenotype is the HIV protease assay described in Example 2. Other assays and methods are useful to the present invention, such as assays determining enzyme structure and function, as well as target/ligand binding and dissociation kinetics including radioligand binding assays, ELISA, mobility shift assays, DNAse hypersensitivity assays, DNA and RNA footprint assays, and the like. Other detection systems include fluorescence resonance emission transfer (FRET), surface plasmon resonance (SPR), protein co-immunoprecipitation, mass spectroscopy including GC-MS, nuclear magnetic resonance including 2-D NMR, and x-ray diffusion crystallography. Structural changes to a bioactive molecule provide a currently preferred method of detecting a phenotype, for example the detection of structural changes to a ribosome in erythromycin resistant E. coli. for (Weisblum, Antimicrobial Agents and Chemotherapy, 39:577-585 (1995) incorporated herein by reference.

Radioligand binding assays can be used to derive and compare equilibrium binding constants (K_{D}) across compound concentration ranges of 1 pM to 10,000 μM, and work with concentrations of bioactive molecules from as little as 10 pMol. The value of K_{D} for a protein and its ligand is related to the IC₅₀, (or the inhibitor concentration displaying 50% inhibition) and can be considered its general equivalent. The change in compound susceptibility can be calculated by comparing the IC₅₀ of the bioactive molecule derived from the patient sample against the IC₅₀ for the wild-type or other acceptable standard. As little as a 1-5% change in relative affinity between the K_{D} values of the wild-type and mutant bioactive molecules can be detected by radioligand binding assays. Any change in K_{D} or IC₅₀ is significant, but a 5% to 10% change in relative affinity indicates a clear decrease in clinical efficacy for a therapeutic compound, while a 50% change indicates a substantial decrease in efficacy, and a 100% change indicates effective loss of binding and effective loss for therapeutic potential, i.e. a drug resistant phenotype.

SPR systems provide assays for monitoring in real time the binding and dissociation of a ligand and its target. These devices can be used to derive and compare equilibrium binding constants (K_{D}) across compound concentration ranges of 0.1 pM to 10,000 μM, and work with concentrations of bioactive molecules from as little as 1 pMol. The change in drug susceptibility can be calculated by comparing the IC₅₀ of the patient sample against the IC₅₀ for the wild-type standard. As little as a 1% change in relative affinity between the K_{D} values of the wild-type and mutant bioactive molecules can be detected by SPR. Any change in K_{D} or IC₅₀ is significant, but a 5% to 10% change in relative affinity indicates a clear decrease in clinical efficacy for a therapeutic compound, while a 50% change indicates a substantial decrease in efficacy, and a 1100% change indicates effective loss of binding and effective loss for therapeutic potential. SPR thus provides an excellent detection system for observing the phenotype of a bioactive molecule.

Commercially available SPR systems include the BIAlite™ and BIAcore™ devices sold by Biacore AB, the IAsys™ device sold by Affinity Sensors Limited (UK), and the BIOS-1 device sold by Artificial Sensor Instruments (Zurich, Switzerland The technical manuals of these systems are hereby incorporated by reference). Displacement or dissociation of, for example, a ligand or drug molecule from a bioactive molecule affixed to the sensor surfaces of such devices causes a relative decrease in mass, which is readily detectable. SPR works best when the net change in mass is large and thus easy to detect. For example, where the drug is a low molecular weight compound, such as a steroid or a peptide, the analogue may be conjugated to a high molecular weight substance so as to create a higher molecular weight difference between the drug and the bioactive peptide. High molecular weight substances suitable for conjugation include proteins such as ovalbumin or bovine serum albumin (BSA), or other entities such as lipids and the like. It is to be noted that these substances are not conventional labels such as enzymes, radiolabels, fluorescent or chemiluminescent tags, redox labels or coloured particles and the like, but serve merely to create a disparity in molecular weight between the drug and its target. Alternatively, where the therapeutic agent is a peptide, the molecular weight of the peptide may be increased relative to the bioactive molecule, by using the peptide as part of a fusion protein. Conveniently the peptide may be fused to the N-terminal or, more preferably, the C-terminal of a polypeptide. Methods for the construction of DNA sequences encoding such fusion proteins are well known to those skilled in the art.

Mass spectroscopy also provides, for example, a means for determining molecular composition, weight, and the presence or absence of candidate binding compounds, thus allowing detection of a phenotype. Mass spectroscopy has the advantage that it can work with femtomolecular concentrations of bioactive molecules. Such devices useful for studing the phenotypes of bioactive molecules include, for example, fast atomic bombardment mass spectrometry (see, e.g., Koster et al., Biomedical Environ. Mass Spec. 14:111-116 (1987)); plasma desorption mass spectrometry; electrospray/ionspray (see, e.g., Fenn et al., J. Phys. Chem. 88:4451-59 (1984), PCT Appln. No. WO 90/14148, Smith et al., Anal. Chem. 62:882-89(1990)); and matrix-assisted laser desorption/ionization (Hillenkamp, et al., “Matrix Assisted UV-LaserDesorption/Ionization: A New Approach to Mass Spectrometry of Large Biomolecules,” Biological Mass Spectrometry (Burlingame and McCloskey, eds.). Elsevier Science Publishers, Amsterdam, pp. 49-60, 1990); Huth-Fehre et al., “Matrix Assisted Laser Desorption Mass Spectrometry of Oligodeoxythymidylic Acids,” Rapid Communications in Mass Spectrometry, 6:209-13 (1992) incorporated by reference).

The assays and methods of the present invention have application in all areas of anti-microbial therapy, such as anti-bacterial therapy, anti-viral therapy and anti-fungal therapy.

Anti-bacterial agents or compounds for use in anti-infective chemotherapy comprise β-lactam antibiotics (e.g., penicillins, cephalosporins, carbapenems, and monobactams), glycopeptides (e.g. vancomycin and teichoplanin) aminoglycoside antibiotics (e.g., kanamycin, gentamicin and amikacin) cephem antibiotics (e.g., cefixime, cefaclor), macrolide antibiotics (e.g., erythromycin), tetracycline antibiotics (e.g., tetracycline, minocycline, streptomycin), quinolone antibiotics, lincosamide antibiotics, trimethoprim, sulfonamides, imipenem, isoniazid, rifampin, rifabutin, rifapentine, pyrazinamide, ethambutol, bismuth salts including bismuth acetate, bismuth citrate, and the like, metronidazole, miconazole, kasugamycin, and quinolone compounds such as ofloxacin, lomefloxacin and ciprofloxacin. These compounds are currently preferred anti-bacterial agents, but new compounds are being developed, which are suitable for use with the assays and methods of the present invention.

Anti-fungal agents or compounds used in anti-infective chemotherapy comprise rapamycin or a rapalog, including e.g. amphotericin B or analogs or derivatives thereof (including 14(s)-hydroxyamphotericin B methyl ester, the hydrazide of amphotericin B with 1-amino-4-methylpiperazine, and other derivatives) or other polyene macrolide antibiotics, including, e.g., nystatin, candicidin, pimaricin and natamycin; flucytosine; griseofulvin; echinocandins or aureobasidins, incluing naturally occurring and semi-synthetic analogs; dihydrobenzo[a]napthacenequinones; nucleoside peptide antifungals including the polyoxins and nikkomycins; allylamines such as naftifine and other squalene epoxidease inhibitors; and azoles, imidazoles and triazoles such as, e.g., clotrimazole, miconazole, ketoconazole, econazole, butoconazole, oxiconazole, terconazole, itraconazole or fluconazole and the like. These compounds are currently preferred anti-fungal agents, but new compounds are being developed, which are suitable for use with the assays and methods of the present invention. For additional conventional anti-fungal agents and new agents under development, see e.g. Turner and Rodriguez, 1996, Recent Advances in the Medicinal Chemistry of Anti-fungal Agents, Current Pharmaceutical Design, 2, 209-224.

Anti-viral agents or compounds used in anti-infective chemotherapy that are suitable for use with the present invention comprise lamivudine, pencyclovir, famcyclovir, adefovir, loviride, aphidicolin, tivirapine, entecavir, clevudine, carbovir, cidofovir, foscarnet, gangcyclovir (GCV), zidovudine (AZT), didanosine (ddI), stavudine (d4T), nevirapine (NVP), delavirdine (DLV), efavirenz (EFN), saquinavir (SQV), indinavir (IDV), ritonavir (RTV), nelfinavir (NFV), abacavir (ABC), arnprenavir (AMP), alpha-interferon, beta-2′,3′-dideoxycytidine (ddC), (±)-2-amino-1,9,dihydro-9-[(1α,3β,4α)-3-hydroxy-4-(hydroxymethyl)cyclopentyl]-6H-purine-6-one (2′-CDG), and 2′,3′-dideoxy-5-fluoro-3′-thiacytidine (FTC), as well as protease inhibitors comprising amprenavir, lopinavir, nelfinavir, ritonavir, KNI-272, as well as therapeutic combinations such as highly active anti-retroviral therapy (HAART). These compounds are currently preferred anti-viral agents, but new compounds are being developed, which are suitable for use with the assays and methods of the present invention, see Squires KE, Antivir Ther, 6 Suppl 3:1-14 (2001) incorporated by reference.

Chemotherapeutic agents or compounds used in anti-infective chemotherapy that are suitable for use with the present invention comprise uracil mustard, chlormethine, cyclophosphamide, fosfamide, melphalan, chlorambucil, pipobroman, triethylenemelamine, triethylenethiophosphoramine, busulfan, carnustine, lomustine, streptozocin, dacarbazine, temozolomide, methotrexate, 5-fluorouracil, floxuridine, cytarabine, 6-mercaptopurine, 6-thioguanine, fludarabine phosphate, pentostatine, gemcitabine, vinblastine, vincristine, vindesine, bleomycin, dactinomycin, daunorubicin, doxorubicin, epirubicin, idarubicin, paclitaxel, mithramycin, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, CPT-11, anastrazole, letrazole, capecitabine, reloxafine, droloxafine, gemcitabine, paclitaxel, and hexamethylmelamine. These compounds are currently preferred anti-cancer agents, but new compounds are being developed, which are suitable for use with the assays and methods of the present invention.

Compounds to treat autoimmune disease states include non-steroidal anti-inflammatories, such as ibuprofen, aspirin, ketoprophen, indomethacin, diclofenac, diflunisal, etodolac, phenoprophen, meclofenamate and the like, including Cox2 specific NSAIDS like celecoxib and rofecoxib, steroids such as prednisone and prednisolone, anti-histamines such as hydroxyzine fexofenidine, cetirazine, loratadine, and diphenhydramine, IL-1 mediators, TNF mediators, Interferon mediators, prostaglandin mediators, anti-rheumatic compounds, and monoclonal antibodies, such as infliximab, basiliximab, pavizumab, and trastuzimab. Antineoplastic agents such as cyclophosphamide, prednisone, levamisol, colchicine, and probenecid are also widely used against autoimmune diseases.

These agents or compounds are generally used in the present invention to contact a bioactive molecule across a concentration range of 0.01-100 times the known IC₅₀ value of the compound and the bioactive molecule. More or less of the compound can be added, for example, to expand the data points defining the inhibition curve, or to define a broad range or dosages where the IC₅₀ value is unknown. The present invention provides an in vitro assay, and the experimental dosage range can be different from dose ranges when these compounds are administered to humans. For example, in vitro a 100-fold increase in drug dosage may be sufficient to eliminate bioactivity of the target compound, but such an extreme dose change would not be permitted in human administration. Human dosages for these compounds are given in the Physician 's Desk Reference (2001) incorporated herein by reference, comprising the phenotype of a bioactive molecule detected by the assays disclosed herein, and a physician or one similarly skilled in the art is capable of viewing experimental data and determining clinical suitability or application. As such, the present invention provides for phenotypic assays and methods of predicting and monitoring a patient's chemotherapy regimen for the above compounds, and for evaluating the potential of newly developed drugs to treat the patient's affliction.

The present invention comprises assays and methods capable of generating sufficient quantities of the desired bioactive molecule for phenotypic detection and characterization in a rapid manner, for example, 24 hours, 48 hours, or approximately one week. Through PCR, LCR, TMA, NASBA, and SDA amplification methods, the target sequence can be amplified in a matter of hours. Using the coupled transcription/translation systems described, protein expression and purification is effectuated in a day. Using the assays described herein, a detection and analysis of the effects of the drug on the functional properties (Phenotype) of its target is achieved within about 24 to 48 hours. This provides a rapid means of evaluating the drug's potential in chemotherapeutic regimens. Examples of additional bioactive molecules appropriate for the present assays and methods disclosed herein as shown in Table 2.

TABLE 2
Drug Resistance and Bioactive Molecules

| ORGANISM | DRUG | PROTEIN | GENE | NCBI ACCESSION No. |
|---|---|---|---|---|
| breast cancer | | antiestrogen | | XP_034007 |
| Homo sapiens | | resistance 1 | | |
| breast cancer | | antiestrogen | | XP_002017 |
| Homo sapiens | | resistance 3 | | |
| Mycoplasma hominis | ciprofloxacin, | DNA Gyrase | gyrA | CAB10849 |
| | ofloxacin, | subunit A | | |
| | lomefloxacin | | | |
| Mycoplasma pneumoniae | KASUGAMYCIN | Dimethyladenosine | | P75113 |
| | | transferase | | |
| herpes simplex virus type | acyclovir | Thymidine | | AAD28536 |
| 1 | | kinase | | |
| herpes simplex virus type | aphidicolin | DNA polymerase | | AAA45854 |
| 2 | | | | |
| herpes simplex virus type | acyclovir | Thymidine kinase | | KIBET3 |
| 2 | | | | |
| hepatitis C virus | interferon | Nonstructural 5A | NS5A | AAB87527 |
| | | protein (induces | | |
| | | interleukin-8) | | |
| Chlamydia trachomatis | quinolone | Gyrase subunit A | gyrA | AF044267 |
| Chlamydia trachomatis | quinolone | Gyrase subunit B | gyrB | AF044267 |
| Chlamydia trachomatis | quinolone | Topoisomerase IV | parC | AF044268 |
| | | subunit A | | |
| Chlamydia trachomatis | quinolone | Topoisomerase IV | parE | AF044268 |
| | | subunit B | | |
| Pasteurella aerogenes | tetracycline | Tetracycline pump | tet(B) gene | AJ278685 |

The following examples as used herein illustrate particular embodiments of the invention described herein.

### EXAMPLE 1

### Hepatitis B (HBV)

HBV is a causative agent for acute and chronic hepatitis, which strikes about 200 million patients worldwide (Zuckerman A. J., Trans. R. Soc. Trop. Med. Hygiene, 76: 711-718 (1982) incorporated by reference). HBV infection acquired in adult life is often clinically inapparent, and most acutely infected adults recover completely from the disease and clear the virus. Rarely, however, the acute liver disease may be so severe that the patient dies of fulminant hepatitis. A small fraction, perhaps 5-10%, of acutely infected adults, becomes persistently infected by the virus and develops chronic liver disease of varying severity. Neonatally transmitted HBV infection, however, is rarely cleared, and more than 90% of such children become chronically infected. Because HBV is commonly spread from infected mother to newborn infant in highly populated areas of Africa and Asia, several hundred million people throughout the world are persistently infected by HBV for most of their lives and suffer varying degrees of chronic liver disease, which greatly increases their risk of developing cirrhosis and hepatocellular carcinoma (HCC). Indeed, the risk of HCC is increased 100-fold in patients with chronic hepatitis, and the lifetime risk of HCC in males infected at birth approaches 40%. Beasley R. P. et al., Lancet (1981) 2, 1129-1133. Incorporated by reference) Accordingly, a large fraction of the world's population suffers from and dies of these late complications of HBV infection. The development of anti-HBV drugs has been long awaited, but has been hampered by the extremely narrow host range of HBV: HBV replicates mainly in human and chimpanzee livers and not in experimental animals or in cultured cells. Tiollais, P et al., Nature (London) (1985) 317, 489-495 incorporated by reference.

Hepatitis B virus is a DNA virus with a compact genomic structure. Despite its small, circular, 3200 base pairs, HBV DNA codes for four sets of viral products and has a complex, multiparticle structure. HBV achieves its genomic economy by relying on an efficient strategy of encoding proteins from four overlapping genes: S, C, P, and X. HBV is one of a family of animal viruses, hepadnaviruses, and is classified as hepadnavirus type 1. Similar viruses infect certain species of woodchucks, ground and tree squirrels, and Peking ducks. All hepadnaviruses, including HBV, share the following characteristics: 1) three distinctive morphological forms exist, 2) all members have proteins that are functional and structural counterparts to the envelope and nucleocapsid antigens of HBV, 3) they replicate within the liver but can also exist in extrahepatic sites, 4) they contain an endogenous DNA polymerase with both RNA- and DNA-dependent DNA polymerase activities, 5) their genomes are partially double stranded circular DNA molecules, 6) they are associated with acute and chronic hepatitis and hepatocellular carcinoma and 7) replication of their genome goes through an RNA intermediate which is reverse transcribed into DNA using the virus's endogenous RNA-dependent DNA polymerase activity in a manner analogous to that seen in retroviruses. In the nucleus of infected liver cells, the partially double stranded DNA is converted to a covalently closed circular double stranded DNA (cccDNA) by the DNA-dependent DNA polymerase. Transcription of the viral DNA is accomplished by a host RNA polymerase and gives rise to several RNA transcripts that differ in their initiation sites but all terminate at a common polyadenylation signal. The longest of these RNAs acts as the pregenome for the virus as well as the message for the some of the viral proteins. Viral proteins are translated from the pregenomic RNAs, and the proteins and RNA pregenome are packaged into virions and secreted from the hepatocyte. Although HBV is difficult to cultivate in vitro, several cells have been successfully transfected with HBV DNA resulting in the in vitro production of HBV particles.

There are three particulate forms of HBV: non-infectious 22 nm particles, which appear as either spherical or long filamentous forms, and 42 nm double-shelled spherical particles which represent the intact infectious hepatitis B virion. The envelope protein, HBsAg, is the product of the S gene of HBV and is found on the outer surface of the virion and on the smaller spherical and tubular structures.

Upstream of the S gene open reading frame are the pre-S gene open reading frames, pre-S 1 and pre-S2, which code for the pre-S gene products, including receptors on the HBV surface for polymerized human serum albumin and the attachment sites for hepatocyte receptors. The intact 42 nm virion can be disrupted by mild detergents and the 27 μm nucleocapsid core . particle isolated. The core is composed of two nucleocapsid proteins coded for by the C gene. The C gene has two initiation codons defining a core and a precore region. The major antigen expressed on the surface of the nucleocapsid core is coded for by the core region and is referred to as hepatitis B core antigen (HBcAg). Hepatitis B e antigen (HBeAg) is produced from the same C gene by initiation at the precore ATG.

Also packaged within the nucleocapsid core is a DNA polymerase, which directs replication and repair of HBV DNA. The DNA polymerase is coded for by the P gene, the third and largest of the HBV genes. The enzyme has both DNA-dependent DNA polymerase and RNA-dependent reverse transcriptase activities and is also required for efficient encapsidation of the pregenomic RNA. The fourth gene, X, codes for a small, non-particle-associated protein which has been shown to be capable of transactivating the transcription of both viral and cellular genes. The DNA polymerase gene was selected as a target in this assay.

Amplification of Human HBV DNA Polymerase

Viral DNA was isolated from HBV patient serum specimens with the QIAamp Blood Kit (Qiagen, Valencia, Calif.). A nested PCR procedure was used to amplify HBV DNA polymerase sequences encoding the wild-type HBV polymerase (HBV-WT), the type-I mutant HBV protein (HBV-M, HM2 and HM5) carrying the mutations L528M and M552V, and the type-II mutants (HM1 and HM3), carrying the mutation M5521. Both mutations are phenotypically associated with lamivudine resistance.

The first-step PCR used primers HB10 (SEQ ID NO:1) and HB11 (SEQ ID NO:2). The second step used primers HB3 (SEQ ID NO:3) and HB6 (SEQ ID NO:4). The reaction mixture in a 50 μl volume for both PCR steps contained 10 mM Tris-HCL, pH 8.3, 50 mM KCL, 1.5 mM MgCl₂, 0.2 mM of each dNTP, 20 pM of each primer, and 1.25 U of Taq DNA polymerase (Perkin Elmer). PCR conditions for both steps were 94° C. for 5 minutes, and then 35 cycles of: 94° C./30 sec., 55° C./1 min., 72° C./3.5 min., followed by a 5 minute extension at 72° C.

The resulting 2.6 kb PCR generated DNA templates contained a T7 RNA polymerase promoter sequence for transcribing the DNA, a Kozak consensus sequence for efficiently translating the RNA, and the specific HBV DNA polymerase sequences from the patient specimens.

Expression of the Polymerase

The PCR-generated DNA templates were directly transcribed and translated in a cell-free expression system into HBV DNA polymerase using a rabbit reticulocyte lysate system, TNT T7 Quick for PCR DNA (Promega, Madison, Wis.). A 90 kDa protein, corresponding to the full length HBV polymerase, was produced from this eukaryotic expression system

Functional Assay for the Polymerase

A sensitive DNA polymerase assay (Roche Molecular Biochemicals, Indianapolis, Ind.) was used to determine the DNA polymerase activity of the expressed HBV polymerase proteins. FIG. 1 measures the DNA dependent DNA polymerase activity of both mutant and wild-type variants of the hepatitis B virus (HBV). The DNA polymerase assay as shown provides a non-radioactive assay, which measures the ability of the enzyme to digoxigenin and biotin labeled nucleotides into freshly synthesized DNA. The detection of synthesized DNA as a parameter for DNA polymerase activity follows a sandwich ELISA protocol—biotin labeled nucleic acid binds the surface of a microtiter plate coated with streptavidin. An anti-digoxigenin antibody conjugated to peroxidase is incubated with the nucleic acid. Upon addition of the peroxidase substrate, a color change occurs corresponding to the peroxidase activity, which is detected by a microplate ELISA reader. The absorbance samples is directly correlated to the level of DNA polymerase activity in the sample. Such an assay is commercially available, for example, the DNA Polymerase, non-radioactive kit, from Roche Molecular Biochemicals. In FIG. 1, HBV-WT refers to the wild-type HBV polymerase. HBV-M refers to an HBV polymerase containing a type-I mutation (L528M and M552V), that is phenotypicaly associated with lamivudine resistance. PC and NC refer respectively to positive and negative controls. Briefly, the positive control includes Klenow enzyme in polymerase buffer. The negative control includes reticulcyte lysate without the DNA amplicon.

FIG. 2 illustrates an inhibition curve of the anti-viral compound lamivudine-TP, and its effects on HBV polymerase activity over a concentration range of the drug. Lamivudine-TP was used to contact the enzyme in the polymerase assay across a final concentration range of 0, 20, 40, 60, 80, 100, 200, and 300 nM. Inhibition of DNA polymerase activity (%) was plotted against compound concentration. Another technique of deriving the IC₅₀ is to plot percent bioactivity against the log of the concentration of the inhibitor drug, in which case the inhibition curve is described by non-linear regression modeling using a single binding site algorithm. Such modeling programs are known in the art and include, for example, PRISM from GraphPad Software, (San Diego, Calif.).

FIG. 3 illustrates an inhibition curve of the anti-viral compound lamivudine-TP, and its effects on wild-type HBV polymerase activity over a concentration range of the drug as against the wild-type HBV polymerase (HBV-WT), the type-I mutant HBV protein (HBV-M, HM2 and HM5), and the type-II mutants (HM1 and HM3, displaying M552I and also phenotypically associated with lamivudine resistance). Lamivudine-TP was added to the polymerase assay across a final concentration range of 0, 60, 100, and 200 nM. Inhibition of DNA polymerase activity (%) was plotted against drug concentration. FIG. 1 illustrates an assay measuring the DNA dependent DNA polymerase activity of both mutant and wild-type variants of the hepatitis B virus (HBV). The DNA polymerase assay as shown provides a non-radioactive assay, which measures the ability of the enzyme to incorporate modified nucleotides into freshly synthesized DNA. The detection of synthesized DNA as a parameter for DNA polymerase activity follows a sandwich ELISA protocol. The absorbence the samples is directly correlated to the level of DNA polymerase activity in the sample. HBV-WT refers to the wild-type HBV polymerase. HBV-M refers to an HBV polymerase containing a type-I mutation (L528M and M552V), that is phenotypicaly associated with lamivudine resistance. PC and NC refer respectively to positive and negative controls.

Interpretation of Phenotype: Drug Susceptibility

The change in drug susceptibility can be calculated by comparing the IC₅₀ of the patient sample by the IC₅₀ for the wild-type standard. As little as a 1%-5% change in relative affinity between the IC₅₀ values of the wild-type and mutant proteins can be detected by this assay. Any change in IC₅₀ is significant, but a 5-10% change in relative affinity indicates a clear decrease in clinical efficacy for a therapeutic compound, while a 50% change indicates a substantial decrease in efficacy suggesting the use of the compound should be discontinued, and a 100% change indicates effectively a complete loss of function. In FIG. 3, the mutant proteins display an IC₅₀ of about 100 nM, while the wild-type polymerase shows an approximate IC₅₀ of about 50 nM, corresponding to a two-fold decrease or 50% reduction in the IC₅₀ value. This corresponds to a drug resistent phenotype in the mutants.

### EXAMPLE 2

### Human Immunodeficiency Virus (HIV)

Acquired immune deficiency syndrome (AIDS) is a fatal human disease, generally considered to be one of the more serious diseases to ever affect humankind. Globally, the numbers of human immunodeficiency virus (HIV) infected individuals and of AIDS cases increase relentlessly and efforts to curb the course of the pandemic, some believe, are of limited effectiveness. Two types of HIV are now recognized: HIV-1 and HIV-2. By Dec. 31, 1994, a total of 1,025,073 AIDS cases had been reported to the World Health Organization. This is only a portion of the total cases, and WHO estimates that as of late 1994, allowing for underdiagnosis, underreporting and delays in reporting, and based on the estimated number of HIV infections, there have been over 4.5 million cumulative AIDS cases worldwide (Mertens et al., (1995) AIDS 9 (Suppl A), S259-S272). Since HIV began its spread in North America, Europe and sub-Saharan Africa, over 19.5 million men, women and children are estimated to have been infected. One of the distinguishing features of the AIDS pandemic has been its global spread within the last 20 years, with about 190 countries reporting AIDS cases today. The projections of HIV infection worldwide by the WHO are staggering. The projected cumulative total of adult AIDS cases by the year 2000 is nearly 10 million. By the year 2000, the cumulative number of HIV-related deaths in adults is predicted to rise to more than 8 million from the current total of around 3 million.

HIV-1 and HIV-2 are enveloped retroviruses with a diploid genome having two identical RNA molecules. The molecular organization of HIV is (5′) U3-R-U5-gag-pol-env-U3-R-U5 (3′). The U3, R, and U5 sequences form the long terminal repeats (LTR) which are the regulatory elements that promote the expression of the viral genes and sometimes nearby cellular genes in infected hosts. The internal regions of the viral RNA code for the structural proteins: gag (p55, p17, p24 and p7 core proteins), pol (p10 protease, p66 and p51 reverse transcriptase and p32 integrase) and env (gp120 and gp41 envelope glycoproteins) Gag codes for a polyprotein precursor that is cleaved by a viral protease into three or four structural proteins; pol codes for reverse transcriptase (RT) and the viral protease and integrase; env codes for the transmembrane and outer glycoprotein of the virus. The gag and pol genes are expressed as a genomic RNA, while the env gene is expressed as a spliced subgenomic RNA. In addition to the env gene, there are other HIV genes produced by spliced subgenomic RNAs that contribute to the replication and biologic activities of the virus. These genes include: tat which encodes a protein that activates the expression of viral and some cellular genes; rev which encodes a protein that promotes the expression of unspliced or single-spliced viral mRNAs; nef which encodes a myristylated protein that appears to modulate viral production under certain conditions; vif which encodes a protein that affects the ability of virus particles to infect target cells but does not appear to affect viral expression or transmission by cell-to-cell contact; vpr which encodes a virion-associated protein; and vpu which encodes a protein that appears to promote the extracellular release of viral particles.

No disease better exemplifies the problem of viral drug resistance than AIDS. Drug resistant HIV isolates have been identified for nucleoside and non-nucleoside reverse transcriptase inhibitors and for protease inhibitors. The emergence of HIV isolates resistant to AZT is not surprising since AZT and other reverse transcriptase inhibitors only reduce virus replication by about 90%. High rates of virus replication in the presence of the selective pressure of drug treatment provide ideal conditions for the emergence of drug-resistant mutants. Patients at later stages of infection who have higher levels of virus replication develop resistant virus with AZT treatment more quickly than those at early stages of infection (Richman et al., (1990) J AIDS 3, 743-6, incorporated by reference). The initial description of the emergence of resistance to AZT identified progressive and stepwise reductions in drug susceptibility (Larder et al., (1989) Science 243, 1731-1734). This was explained by the recognition of multiple mutations in the gene for reverse transcriptase that contributed to reduced susceptibility (Larder et al., (1989) Science 246, 1155-1158, incorporated by reference). These mutations had an additive or even synergistic contribution to the phenotype of reduced susceptibility (Kellam et al., (1992) Proc. Natl. Acad. Sci. 89, 1934-1938). The cumulative acquisition of such mutations resulted in progressive decreases in susceptibility. Similar effects have been seen with non-nucleoside reverse transcriptase inhibitors (Nunberg et al., (1991) J Virol 65, 4887-4892; Sardanna et al., (1992) J Biol Chem 267, 17526-17530, incorporated by reference). Studies of protease inhibitors have found that the selection of HIV strains with reduced drug susceptibility occurs within weeks (Ho et al., (1994) J Virol 68, 2016-2020; Kaplan et al., (1994) Proc. Natl. Acad. Sci. 91, 5597-5601). While recent studies have shown protease inhibitors to be more powerful than reverse transcriptase inhibitors, nevertheless resistance has developed. (Condra et al., Id. and Report 3rd Conference on Retroviruses and Opportunistic Infections, March 1996, incorporated by reference). Subtherapeutic drug levels, whether caused by reduced dosing, drug interactions, malabsorption or reduced bioavailability due to other factors, or self-imposed drug holidays, all permit increased viral replication and increased opportunity for mutation and resistance.

The selective pressure of drug treatment permits the outgrowth of preexisting mutants. With continuing viral replication in the absence of completely suppressive anti-viral drug activity, the cumulative acquisition of multiple mutations can occur over time, as has been described for AZT and protease inhibitors of HIV. Indeed viral mutants multiply resistant to different drugs have been observed (Larder et al., (1989) Science 243, 1731-1734; Larder et al., (1989) Science 246, 1155-1158; Condra et al., (1995) Nature 374, 569-71). With the inevitable emergence of resistance in many viral infections, as with HIV for example, strategies must be designed to optimize treatment in the face of resistant virus populations. Ascertaining the contribution of drug resistance to drug failure is a difficult problem because patients who are more likely to develop drug resistance are more likely to have other confounding factors that will predispose them to a poor prognosis (Richman (1994) AIDS Res Hum Retroviruses 10, 901-905). In addition patients contain mixtures of viruses with different susceptibilities.

Isolation and Amplification of the HIV Proteins

A phenotypic assay for assessment of drug susceptibility of HIV Type 1 isolates to reverse transcriptase (RT) inhibitors has been developed. This method provides the physician with information as to whether to continue with the existing chemotherapeutic regimen or to alter the therapy. Viral load monitoring is becoming a routine aspect of HIV care. However, viral load number alone cannot be used as a basis for deciding which drugs to use alone or in combination. Combination therapy is becoming increasingly the chemotherapeutic regimen of choice. When a person using a combination of drugs begins to experience drug failure, it is impossible to know with certainty, which of the drugs in the combination is no longer active. One cannot simply replace all of the drugs, because of the limited number of drugs currently available. Furthermore, if one replaces an entire chemotherapeutic regimen, one may discard one or more drugs that are active for that particular patient. Also, it is possible for viruses that display resistance to a particular inhibitor to also display varying degrees of cross-resistance to other inhibitors. Ideally, therefore, every time a person has a viral load test and a viral load increase is detected, the drug sensitivity/resistance assay of the present invention should also be carried out. Until effective curative therapy is developed, management of HIV disease will require such testing.

The sequence of HIV-1 (isolate HXB2, reference genome, 9718 bp) was obtained from the National Center for Biotechnology Information (NCBI), National Library of Medicine, National Institutes of Health via the ENTREZ Document Retrieval System (Genbank name: HUVHXB2CG, Genbank Accesion No: 0/3455). Primer sets are developed, which are designed to amplify the gene of interest. In the case where the sequence to be reverse transcribed is that coding for reverse transcriptase or reverse transcriptase and protease, the downstream primer is preferably a combination of OUT 3 (downstream) and RVP 5 (upstream), the OUT 3 primer comprising 5′-CAT TGC TCT CCA ATT ACT GTG ATA TTT CTC ATG-3′ (SEQ ID NO:5) and RVP 5 comprising sequence 5′-GGG AAG ATC TGG CCT CCT ACA AGG G-3′ (SEQ ID NO:6) using the PCR conditions as described in Maschera, B., et al. Journal of Virology, 69, 5431-5436.

The desired sequence from the pol and RT genes are isolated from a sample of a biological material obtained from the patient whose phenotypic drug sensitivity is being determined. A wide variety of biological materials can be used for the isolation of the desired sequence. The biological material can be selected from plasma, serum or a cell-free body fluid selected from semen and vaginal fluid. Plasma is particularly preferred and is particularly advantageous. When a biological material such as plasma is used in the isolation of the desired sequence, a minimal volume of plasma can be used, typically about 50-500 μl, more particularly of the order of 200 μl. Alternatively, the biological material can be whole blood to which an RNA stabilizer has been added. In a still further embodiment, the biological material can be a solid tissue material selected from brain tissue or lymph nodal tissue, or other tissue obtained by biopsy. Viral RNA is conveniently isolated in accordance with the invention by methods known per se, for example the method of Boom, R. et al., Journal of Clinical Microbiology, 28:3, 495-503 (1990); in the case of plasma, serum and cell-free body fluids, one can also use the QIAamp viral RNA kit marketed by the Qiagen group of companies.

Reverse transcription can be carried out with a commercial kit such as the GeneAmp Reverse Transcriptase Kit marketed by Perkin Elmer. The desired region of the patient pol gene is preferably reverse transcribed using a specific downstream primer. In a particularly preferred embodiment a patient's HIV RT gene and HIV protease gene are reverse transcribed using the HIV-1 specific OUT 3 primer and a genetically engineered reverse transcriptase lacking RNase H activity, such that the total RNA to be transcribed is converted to cDNA without being degraded. Such a genetically engineered reverse transcriptase, the Expand (Expand is a Trade Mark) reverse transcriptase, can be obtained from Boehringer Mannheim GmbH. Expand reverse transcriptase is a RNA directed DNA polymerase. The enzyme is a genetically engineered version of the Moloney Murine Leukemia Virus reverse transcriptase (M-MuLV-RT). Point mutation within the RNase H sequence reduces the RNase H activity to below the detectable level. Using this genetically engineered reverse transcriptase enables one to obtain higher amounts of full length cDNA transcripts. Following reverse transcription the transcribed DNA is amplified using the technique of PCR, and preferably the product of reverse transcription is amplified using a nested PCR technique. Preferably, in the case where the region of interest is the RT region, a nested PCR technique is used using inner and outer primers as described by Kellam, P. and Larder, B. A., Antimicrobial Agents and Chemotherapy, 38:1, 23-30 (1994).

Expression of the HIV Proteins

The PCR-generated DNA templates were expressed into HIV reverse transcriptase and protease using a coupled reticulocyte lysate system, TNT T7 Quick for PCR DNA (Promega, Madison, Wis.). Sizes of the proteins, as well as a confirmation of their integrity, was confirmed by Western Blot.

Functional Assay for the HIV Proteins

The protein is used in inhibition assays with one or more of the following compounds:

RT inhibitors such as AZT, ddI (didanosinenidex (Videx is a Trade Mark), ddC (zalcitabine), 3TC (lamivudine), d4T (stavudine), non-nucleoside RT inhibitors such as delavirdine (U 9051125 (BMAP)/Rescriptor (Rescriptor is a Trade Mark)), loviride (alpha-APA), nevirapine (B1-RG-587/Viramune (Viramune is a Trade Mark) and tivirapine (8-Cl-TIBO(R86183)), and protease inhibitors such as saquinavir, indinavir and ritonavir. These inhibitors are added to protein samples in a nucleoside incorporation assay or protease activity assay as described, contacting the bioactive molecule across a concentration range of 1.0 pM to 10,000 μM thereby generating an IC₅₀ value as described for the wild-type and patient-derived proteins.

A homogeneous time-resolved fluorescence (HTRF) assay has been developed for human immunodeficiency viral (HIV) protease. The assay utilizes a peptide substrate, differentially labeled on either side of the scissile bond, to bring two detection components, streptavidin-cross-linked XL665 (SA/XL665) and a europium cryptate (Eu(K))-labeled antiphosphotyrosine antibody, into proximity allowing fluorescence resonance energy transfer (FRET) to occur. Cleavage of the doubly labeled substrate by HIV protease precludes complex formation, thereby decreasing FRET, and allowing enzyme activity to be measured. The reaction conditions were as described in Cummings RT, et al., Anal Biochem April 10;269(1):79-93 (1999), incorporated by reference. Examination of the first-order rate constant versus enzyme concentration suggests a K_{D} value for the HIV protease monomer-dimer equilibrium. The FRET assay was also utilized to measure the inhibition of the HIV protease enzyme in the presence of anti-viral compounds (see, Cummings RT).

Interpretation of Phenotype: Drug Susceptibility

The relative difference in IC₅₀ value between the patient derived protein and the wild-type protein indicates a potential difference in the effectiveness of the anti-viral agent. For example, a patient diagnosed as being afflicted with HIV undergoes the assay of the present invention. The patient is undergoing combination chemotherapy with the anti-viral agents ddI and AZT. Phenotype testing indicates the IC₅₀ value for the anti-viral agent ddI is 50 nM when tested against the wild-type protein, and 47 nM when tested against the patient sample. This approximate equivalence suggests that the HIV infection under investigation has not developed resistance to ddI. In contrast, the IC₅₀ value for AZT is 1.0 nM when tested against the wild-type protein, and 4.7 nM when tested against the patient sample. An approximate five-fold difference in the IC₅₀ value suggests the infection is developing resistance to AZT. However, the compound lamivudine is considered as a candidate therapeutic agent. The IC₅₀ value for lamivudine is 30.0 nM when tested against the wild-type protein, and 15 nM when tested against the patient sample. The two-fold difference in the IC₅₀ values suggests that lamivudine as an appropriate therapeutic agent. The patient's physician or one similarly skilled in the art uses the relative the IC₅₀ values of the drugs to determine that lamivudine and AZT provide the best combination of anti-viral agents, and that ddI administration should be discontinued.

### EXAMPLE 3

### Hepatitis C Virus (HCV)

Hepatitis C virus (HCV) infection occurs throughout the world and, prior to its identification, represented the major cause of transfusion-associated hepatitis. The seroprevalence of anti-HCV in blood donors from around the world has been shown to vary between 0.02% and 1.23%. HCV is also a common cause of hepatitis in individuals exposed to blood products. There have been an estimated 150,000 new cases of HCV infection each year in the United States alone during the past decade (Alter, Infect. Agents Dis. 2, 155-166 (1993); Houghton 1996, in Fields Virology, 3rd Edition, pp. 1035-1058, hereby incorporated by reference).

The hepatitis C virus (HCV) is a member of the flaviviridae family of viruses, which are positive stranded, non-segmented, RNA viruses with a lipid envelope. Other members of the family are the pestiviruses (e.g., bovine viral diarrheal virus, or BVDV, and classical swine fever virus, or CSFV), and flaviviruses (e.g., yellow fever virus and Dengue virus). See Rice, 1996 in Fields Virology, 3rd Edition, pp. 931-959). Molecular dissection of HCV replication and hence understanding the functions of its encoded proteins, while greatly advanced by the isolation of the virus and sequencing of the viral genome, has been hampered by the lack of an efficient cell culture system for production of native or recombinant HCV from molecular clones. However, low-level replication has been observed in several cell lines infected with virus from HCV-infected humans or chimpanzees, or transfected with RNA derived from cDNA clones of HCV.

HCV replicates in infected cells in the cytoplasm, in close association with the endoplasmic reticulum. Incoming positive sense RNA is released and translation is initiated via an internal initiation mechanism (Wang et al., J. Virol. 67, 3338-3344 (1993) and Tsukiyama-Kohara et al., J. Virol. 66, 1476-1483(1992), hereby incorporated by reference). Internal initiation is directed by a cis-acting RNA element at the 5′ end of the genome; some reports have suggested that full activity of this internal ribosome entry site, or IRES, is seen with the first 700 nucleotides, which spans the 5′ untranslated region (UTR) and the first 123 amino acids of the open reading frame (ORF) (Lu and Wimmer, PNAS 93, 1412, (1996) hereby incorporated by reference). All of the protein products of HCV are produced by proteolytic cleavage of a large (3010-3030 amino acids, depending on the isolate) polyprotein, carried out by one of three proteases: the host signal peptidase, the viral self-cleaving metalloproteinase, NS2, or the viral serine protease NS3/4A. The combined action of these enzymes produces the structural proteins (C, E1 and E2) and non-structural (NS2, NS3, NS4A, NS4B, NS5A, and NS5B) proteins which are required for replication and packaging of viral genomic RNA. NS5B is the viral RNA-dependent RNA polymerase (RDRP) that is responsible for the conversion of the input genomic RNA into a negative stranded copy (complimentary RNA, or cRNA); the cRNA then serves as a template for transcription by NS5B of more positive sense genomic/messenger RNA.

Several institutions and laboratories are attempting to identify and develop anti-HCV drugs. Currently, the only effective therapy against HCV is alpha-interferon, which can control the amount of virus in the liver and blood (viral load) in only a small proportion of infected patients (Houghton 1996, in Fields Virology, 3rd Edition, pp. 1035-1058 and Chung R T, et al., Proc Natl Acad Sci USA August 14;98(17):9847-52 (2001) incorporated by reference). However, given the availability of the molecular structure of the HCV serine protease, NS3/4A (Love et al., Cell 87, 331-342 (1996); Kim et al., Cell 87, 343-355 (1996) hereby incorporated by reference), and success using protease inhibitors in the treatment of HIV-1 infection, there should soon be alternatives available. In addition to HCV protease inhibitors, other inhibitors that might specifically interfere with HCV replication could target virus specific activities such as internal initiation directed for example, by the IRES, RDRP activity encoded by NS5B, or RNA helicase activity encoded by NS3.

As a result of a high error rate of their RDRPs, RNA viruses are particularly able to adapt to many new growth conditions. Most polymerases in this class have an estimated error rate of 1 in 10,000 nucleotides copied. With a genome size of approximately 9.5 kb, at least one nucleotide position in the genome of HCV is likely to sustain a mutation every time the genome is copied. It is therefore likely for drug resistance to develop during chronic exposure to an anti-viral agent. As in the case of HIV, a rapid and convenient assay for drug resistant HCV would greatly improve the likelihood of successful antiviral therapy, given a selection of drugs and non-overlapping patterns of drug resistant genotypes. Resistance-associated mutations can sometimes be identified rapidly by growing the virus in cell culture in the presence of the drug, an approach used with considerable success for HIV-1. To date, however, a convenient cell culture system for HCV is lacking. Therefore, it is not possible to determine the precise nature of genetic changes that confer a drug resistant phenotype in vitro. Thus, in the absence of a database of known resistance-associated mutations, the preferred resistance assay is one that relies on a phenotypic readout rather than a genotypic one. The present invention provides an assay and method for evaluating a compound's effect on a bioactive molecule expressed by the hepatitis C virus, where the virus is obtained from patient samples.

Popular targets for anti-HCV therapy include the host signal peptidase, the viral self-cleaving metalloproteinase, NS2, or the viral serine protease NS3/4A. The combined action of these enzymes produces the structural proteins (C, E1 and E2) and non-structural (NS2, NS3, NS4A, NS4B, NS5A, and NS5B) proteins which are required for replication and packaging of viral genomic RNA. NS5B is the viral RNA-dependent RNA polymerase (RDRP) that is responsible for the conversion of the input genomic RNA into a negative stranded copy. According to the methods of the present invention, the HCV bioactive molecule NS5B is amplifed in vitro and expressed in vitro. The NS5B protein encoded by the amplified nucleic acid sequence is a functioning RNA-dependent RNA polymerase (RdRp), that can be assayed for polymerase activity in the presence and absence of compounds either known to inhibit polymerase activity or compounds under discovery for such properties. Resistance phenotypes are detected by measuring a change in the RNA-dependent RNA polymerase activity of the patient derived recombinant NS5B protein in the presence and absence of the inhibitory compound.

Amplification of the HCV NS5B Gene

Patient blood samples yielded patient derived hepatitis C virus. The sequence of wild-type HCV, isolate: JPUT971017, reference genome hepatitis C virus, 1773 bp) was obtained from the National Center for Biotechnology Information (NCBI), National Library of Medicine, National Institutes of Health via the ENTREZ Document Retrieval System (Genbank Accession No: 9757541 (see also, Murakami,K., et al., Arch. Virol. 146 (4), 729-741 (2001) and Kato N, et al., Proc. Natl. Acad. Sci USA, 87:9524 (1990), hereby incorporated by reference. Primer sets are developed, designed to amplify the NS5B RNA-dependent RNA polymerase gene, encoded at bases 7668 to 9440. Examples of such primer sets and PCR amplification conditions for the NS5B gene are given in Ding J, et al., Chin Med J (Engl) Feb; 111(2): 128-31 (1998) and Holland PV et al., J Clin Microbiol., Oct;34(10):2372-8(1996) hereby incorporated by reference.

Expression of the HCV Proteins

The PCR-generated DNA template were directly transcribed and translated in vitro into HCV NS5B protein using a coupled reticulocyte lysate system, TNT T7 Quick for PCR DNA (Promega, Madison, Wis.). A 65 kDa protein, corresponding to the full length HCV NS5B protein, was produced from this eukaryotic expression system. Size and integrity of HCV NS5B was confirmed by Western Blot.

Functional Assay for the HCV Proteins

An RNA polymerase assay, designed to measure the ability of the enzyme to incorporate modified nucleotides into freshly synthesized RNA, is used to characterize the ability of several anti-viral agents to inhibit the NS5B polymerase. The detection of synthesized RNA provides the parameter for viral RNA-dependent RNA polymerase (RDRP) activity, and follows the methods of Zhong W., et al., J Virol Feb;74(4):2017-22 (2000); Lohmann V, et al., J Viral Hepat May;7(3):167-74 (2000); Ferrari E, et al., J Virol Feb;73(2):1649-54 (1999); Ishii K, et al., Hepatology Apr;29(4):1227-35 (1999); Behrens S E, et al., EMBO J January 2;15(1):12-22 (1996); Zhong W J, et al., Virol Oct;74(19):9134-43 (2000); and Oh J W, et al., J Biol Chem June 9;275(23):17710-7 (2000) incorporated by reference.

The NS5B protein is used in inhibition assays with one or more of the following compounds: viral inhibitors such as AZT, ddI (didanosine/Videx®, ddC (zalcitabine), 3TC (lamivudine), d4T (stavudine), ribavirin triphosphates, non-nucleoside RT inhibitors such as delavirdine (U 9051125 (BMAP)/Rescriptor®, loviride (alpha-APA), nevirapine (B1-RG-587/Viramune® and tivirapine (8-Cl-TIBO(R86183), and gliotoxin). These inhibitors are added to NS5B protein samples across a concentration range of 1.0 pM to 10,000 μM thereby generating an IC₅₀ value as described for the compound and both the wild-type and patient-derived NS5B proteins (see, Zhong W., Ishii K., and Lohmann V., supra).

Interpretation of Phenotype: Drug Susceptibility

By analysing a series of nucleosidic and non-nucleosidic compounds for their effect on RNA dependent RNA polymerase (RdRp) activity, we found, for example, that ribavirin triphosphates have no inhibitory effect on either the wild-type or patient derived NS5B protein, while gliotoxin, a known poliovirus 3D RdRp inhibitor in poliovirus, inhibited HCV NS5B RdRp of both wild-type and patient derived proteins in a dose-dependent manner. The change in drug susceptibility can be calculated by comparing the IC₅₀ of the patient sample by the IC₅₀ for the wild-type standard. As little as a 1%-5% change in relative affinity between the IC₅₀ values of the wild-type and mutant proteins can be detected by this assay. The change in affinity indicates a drug resistant phenotype that is used to determine future chemotherapy regimens.

### EXAMPLE 4

### Human Cytomegalovirus (HCMV)

Human cytomegalovirus (HCMV) is endemic throughout the world and infection rates appear to be relatively constant throughout the year rather than seasonal. Humans are the only known reservoir for HCMV and natural transmission occurs by direct or indirect person-to-person contact. Between 0.2% and 2.2% of infants born in the United States are infected in utero. Another 8 to 60% become infected during the first six months of life as a result of infection acquired during birth or following breast feeding. Because of the high incidence of reactivation of HCMV infection in the breast, breast milk transmission could represent the most common mode of HCMV transmission worldwide. In most developed countries, 40% to 80% of children are infected before puberty. In other areas of the world, 90% to 100% of the population become infected during childhood.

Human cytomegalovirus (HCMV) is a member of the herpesvirus family. A typical herpes virion consists of a core containing a linear double-stranded DNA and icosadeltahedral capsid approx. 100-110 nm in diameter containing 162 capsomeres with a hole running down the long axis, an amorphous “integument” that surrounds the core and an envelope containing viral glycoprotein spikes on its surface. Virion sizes range from 120-300 μm due to differences in the thickness of the tegument layer. There are three subgroups of herpesviruses:

1. Alphaherpesvirinae: HSV, VZV. variable host range, relatively short reproductive cycle, rapid spread in culture, efficient destruction of infected cells, capacity to establish latent infections in sensory ganglia.

2. Betaherpesvirinae: HCMV. Restricted host range, long reproductive cycle, slow progression of infection in culture. Infected cells become enlarged and carrier cells are readily established. Virus can be maintained in latent form in secretory glands, lymphoreticular cells, kidneys and other tissues.

3. Gammaherpesvirinae: EBV. experimental host range extremely narrow, replicate in lymphoblastdid cells and cause lytic infections in some types of epithelial and fibroblastoid cells.

There are eight known human herpesviruses: Human herpesvirus 1 (Herpes simplex virus 1, HSV-1), Human herpesvirus 2 (Herpes simplex virus 2, HSV-2), Human herpesvirus 3 (Varicella-zoster virus, VZV), Human herpesvirus 4 (Epstein-Barr virus, EBV), Human herpesvirus 5 (Human cytomegalovirus), Human herpesvirus 6, Human herpesvirus 7, and Human herpesvirus 8. The genomes of herpes viruses consist of a linear double-stranded (ds) DNA in the virion that circularizes and concatamerizes upon release from the virus capsid in the nucleus of infected cells. The genomes of herpesviruses range in size from 120 to 230 kilobase pairs (kbp). The genomes are polymorphic in size (up to 10 kbp differences) within an individual population of virus. This variation is due to the presence of terminal and internal reiterated sequences. Herpes viruses can be classified into six groups, A through F, based on their overall genome organization. HSV and HCMV fall into group E, in which sequences from both termini are repeated in an inverted orientation and juxtaposed internally, dividing the genomes into two components, L(long) and S(short), each of which consists of unique sequences, UL and Us, flanked by inverted repeats. In these viruses both components can invert relative to each other and DNA extracted form virions consists of four equimolar populations differing in the relative orientation of the two components.

HCMV is a betaherpesvirus and is unique among the betaherpesvirinae in that it falls into the class E genome type. The genome of HCMV is approximately 230 kbp in length and has been completely sequenced (EMBL Seq database accession #X17403). In a naturally occurring population of virus, the genome exists in four isomers. In HCMV, as in HSV, the L-S junction can be deleted, thereby freezing the genome in one of four isomers without dramatically affecting the ability of the virus to grow in cultured cells.

The HCMV genome contains terminal repeat sequences “a” and “a′” present in a variable number in direct orientation at both ends of the linear genome. A variable number of “a” repeats are also present in an inverted orientation at the L-S junction. The number of “a” sequences in these locations ranges from 1-10 with 1 predominating. The size of “a” in HCMV ranges from 700-900 bp. The “a” sequence carries the cleavage and packaging signal. The packaging signals are two highly conserved short sequence elements located within “a” designated pac-1 and pac-2. A 220-bp fragment that carries both the pac-1 and pac-2 elements is sufficient to convey sites for cleavage/packaging as well as inversion on a recombinant CMV construct. The termini of the linear genome are generated by a cleavage event that leaves a single 3′ overhanging nucleotide at either end of the genome. The genome is further characterized by large inverted repeats called “b” and “b′” (or TRL and IRL) and “c” and “c′” (or IRS and TRS) that flank unique sequences UL and Us, that make up the L and S components of the genome.

The HCMV replication cycle is relatively slow compared to other herpesviruses. Viral replication involves the ordered expression of consecutive sets of viral genes. These sets are expressed at different times after infection and include the alpha (immediate early), beta1 and beta2 (delayed early), and gamma 1 and gamma 2 (late) sets based on the time after infection that their transcripts accumulate. DNA replication, genome maturation and virion morphogenesis are coordinated through the temporal regulation of the appropriate gene products required for each step. Expression of gene products is rapid. Late gene expression is delayed for 24-36 hours. Progeny virions begin to accumulate 48 hours post-infection and reach maximal levels at 72-96 hours. In permissive fibroblasts, DNA replication can be detected as early as 14-16 hours post-infection. HCMV stimulates host DNA, RNA and protein synthesis. HCMV replicates more rapidly in actively dividing cells and HCMV replication is inhibited by pretreating cells with agents that reduce host cell metabolism. The HCMV genome circularizes soon after infection. Circles give rise to concatamers and genomic inversion occurs within concatameric forms of the DNA. The majority of replicating DNA is larger than unit length and lacks terminal fragments based on southern blot analysis.

Targets for Drug Resistance

The drugs currently used to treat HCMV (ganciclovir (GCV), foscamet, cidofovir) are known to select for mutations in two viral genes, the UL97 phosphotransferase and the UL54 viral DNA polymerase. GCV-resistant HCMV has been recovered from the central nervous system (CNS) of patients with HCMV-associated neurologic disease who had received long-term GCV maintenance therapy. Resistant strains of HCMV may be selected and preferentially located in the CNS. It is frequently not possible to culture virus from the cerebral spinal fluid (CSF) but it is possible to amplify HCMV DNA using PCR.

Primary isolates of CMV may replicate slowly. In addition, there is a marked delay in the growth rate of some of the drug resistant clinical isolates. In a mixed virus population, a resistant virus population could be masked by a sensitive one. Thus assay results that depend on the growth of virus could be unreliable. Most assays for viral culture use blood or urine, because they are easy to obtain. However, the virus from these compartments may not represent the virus in specific tissues where disease is occurring (especially vitreous fluid and csf). Although there are a few amino acid residues that are modified relatively frequently among drug-resistant strains of herpesviruses recovered from patients, the broad distribution of mutations in the majority of strains makes rapid genetic screening methods impractical. Importantly, since the drug-susceptibility phenotypes resulting from individual genetic changes are complex and variable, a biological test for anti-viral susceptibility of HCMV would be more informative.

UL97: Mutations associated with GCV resistance include amino acids: 460, 520, 590, 591, 592, 593, 594, 595, 596, 600, 603, 607, 659, and 665. The phosphotransferase protein has two functional domains, 1) the amino terminal 300 amino acids code for a regulatory domain and 2) the carboxy terminal 400 amino acids define the catalytic domain. All known drug-resistance mutations are found in the catalytic domain (approx 1.2 kb of sequence). In HSV, the thymidine kinase gene product (TK) is responsible for the phosphorylation of GCV in cells and resistance to GCV in HSV is associated with mutations in the thymidine kinase gene. HCMV has no homolog to the HSV thymidine kinase gene. The gene homologous to UL97 in HSV (UL13) is a protein kinase.

UL54: Mutations in this gene can result in resistance to GCV and other nucleoside analogs (including cidofovir) as well as foscarnet. Mutations associated with foscarnet resistance include amino acid numbers: 700 and 715. Mutations associated with GCV resistance include amino acid numbers: 301, 412, 501, 503, and 987. The mature protein has four recognized domains: 1) a 5′-3′ exoRNAse H. a 3′-5′ exonuclease, a proposed catalytic domain and an accessory protein binding domain. New therapies in development include agents targeted to the CMV protease (UL80) and the DNA maturational enzyme (“terminase”), see, Mousavi-Jazi M et al., J Clin Virol Dec;23(1-2):1-15 (2001) and Jabs, D. A., et al., J Infect Dis, January 15;183(2):333-337 2001) incorporated herein by reference.

Amplification of the CMV Gene of Interest

The sequence of HSV-6 reference genome human herpesvirus 6, was obtained from the National Center for Biotechnology Information (NCBI), National Library of Medicine, National Institutes of Health via the ENTREZ Document Retrieval System (Genbank Accession No.:NP/042935 (see also, Kato N, et al., Proc. Natl. Acad. Sci USA, 87:9524 (1990) and Teo, I. A., et al., Journal of Virology. 65 (9), 4670-4680 (1991) incorporated by reference. Primer sets are developed, which are designed to amplify the UL97 and UL54 genes.

Expression of the CMV Proteins

The PCR-generated DNA templates were directly transcribed and translated in a cell-free system using a coupled reticulocyte lysate system, TNT T7 Quick for PCR DNA (Promega, Madison, WI). Size and integrity of the proteins was confirmed by Western Blot.

Functional Assay for the CMV Proteins

An phosphatase assay designed to measure the ability of the UL97 enzyme to catalyze the trasfer of phosphate was developed. A polymerase assay designed to measure the ability of UL54 to polymerize nucleic acids was also developed. The assays are described herein. The protein is used in inhibition assays with one or more of the following compounds: viral inhibitors such as AZT, ddI (didanosine/Videx (Videx is a Trade Mark), ddC (zalcitabine), 3TC (lamivudine), d4T (stavudine), non-nucleoside RT inhibitors such as delavirdine (U 9051125 (BMAP)/Rescriptor (Rescriptor is a Trade Mark)), loviride (alpha-APA), nevirapine (BI-RG-587/Viramune (Viramune is a Trade Mark) and tivirapine (8-Cl-TIBO(R86183)), and protease inhibitors such as saquinavir, indinavir and ritonavir. These inhibitors are added to protein samples in a nucleoside incorporation assay or protease activity assay as described across a concentration range of 1.0 pM to 10,000 μM thereby generating an IC₅₀ value as described for the wild-type and patient-derived proteins.

Interpretation of Phenotype: Drug Susceptibility

The change in drug susceptibility can be calculated by comparing the IC₅₀ of the patient sample by the IC₅₀ for the wild-type standard. As little as a 1%-5% change in relative affinity between the IC₅₀ values of the wild-type and mutant proteins can be detected by this assay. Any change in IC₅₀ is significant, but a 5-10% change in relative affinity indicates a clear decrease in clinical efficacy for a therapeutic agent, while a 50% change indicates a substantial decrease in efficacy suggesting the use of the compound should be discontinued, and a 100% change indicates effectively a complete loss of therapeutic potential.

### EXAMPLE 5

Autoimmune Disorders

A variant allele of Poly(ADP-ribosyl) transferase (PARP) is diagnostic of systemic lupus erythomatosis (SLE) in a subject having clinical SLE symptoms, or indicates a genetic predisposition for developing SLE in a subject who does not present SLE symptoms (see, U.S. Pat. No. 6,280,941). Poly(ADP-ribosyl) transferase (E.C. 2.4.2.30) functions in the maintenance of genomic integrity; it is the only enzyme known to synthesizes ADP-ribose polymers from nicotinamide adenine dinucleotide (NAD+) and is activated in response to DNA strand breaks. (W. M. Shieh, et al., J. Biol. Chem. 273:30069-72 (1998) incorporated herein by reference. Poly(ADP-ribosyl) transferase enzyme has been shown to stimulate DNA polymerase a by physical association and may form a complex with DNA polymerase alpha in vivo. (Simbulan, C M et al., J. Biol. Chem. 268:93-99 (1993) incorporated herein by reference. Activation of poly(ADP-ribosyl) transferase requires both the DNA-binding capacity of the DNA-binding domain (“zinc fingers”) and the ability to maintain a conformation of the DNA-binding domain that can transfer an “activation signal” to the catalytic domain of the enzyme (Trucco, et al., FEBS Lett. 399:313-16 (1996) incorporated herein by reference).

The important physiologic function of poly(ADP-ribosyl) transferase has been extensively studied by using specific inhibitors (3-aminobenzamide, 3-methoxybenzamide, or antisense RNA) and by studies of knockout mice. (Jeggo, Pa., et al., Current Biol. 8:49-5 (1998) incorporated herein by reference. Cumulative data have shown that the absence of poly(ADP-ribosyl) transferase activity results in elevated spontaneous genetic rearrangements and hypersensitive responses to DNA damage, implying a substantial role for poly(ADP-ribosyl) transferase in maintaining genomic stability. Although no gross defects in apoptosis are found in PARP knockout mice, splenocytes of these mice display a more rapid apoptotic response to an alkylating agent. Cell lines with disrupted PARP expression show insensitivity to apoptotic signals. (Simbulan-Rosenthal C M, et al., J. Biol. Chem. 273:13703-12 (1998) incorporated herein by reference). While PARP has a regulatory role in induced apoptosis, impaired apoptosis is less detectable in whole animals than in cell lines, probably because of other compensatory routes within the organism.

Amplification and expression of PARP are effectuated as described. PARP activity is detected by its ability to bind p53 protein. The binding can be detected by co-immunoprecipitation. Using SPR as described, the affinity of a compound for PARP can be derived.

Interpretation of Phenotype: Drug Susceptibility

The change in drug susceptibility can be calculated by comparing the IC₅₀ of the patient sample by the IC₅₀ for the wild-type standard. As little as a 1%-5% change in relative affinity between the IC₅₀ values of the wild-type and mutant proteins can be detected by this assay. Any change in IC₅₀ is significant, but a 5-10% change in relative affinity indicates a clear decrease in clinical efficacy for a therapeutic agent, while a 50% change indicates a substantial decrease in efficacy suggesting the use of the compound should be discontinued, and a 100% change indicates effectively a complete loss of therapeutic potential.

### EXAMPLE 6

Bacterial Resistance to Quinolone Compounds

Fluoroquinolones are broad-spectrum and effective antibiotics for the treatment of bacterial infections. The primary targets of fluoroquinolone are DNA gyrase and topoisomerase IV, which alter DNA topology through a transient double-stranded DNA break. DNA gyrase is composed of GyrA and GyrB subunits, which are encoded by gyrA and gyrB genes, respectively. Topoisomerase IV includes ParC and ParE subunits, which are encoded by parC and parE genes, respectively. Mutations in the quinolone resistance-determining region (QRDR), primarily the gyrA gene or the parC gene, are associated with quinolone resistance. Mutations in the QRDR of gyrB gene or parE gene are also believed to play a role in quinolone resistance, albeit to a lesser extent. DNA gyrase appears to be the primary quinolone target for gram-negative bacteria, while topoisomerase IV appears to be the preferential target in gram-positive organisms. Mutations in DNA gyrase and/or topoisomerase IV genes are frequently encountered in quinolone-resistant mutants of Streptococcus pneumoniae and Staphylococcus aureus, for example, fluoroquinolone-resistant cultures of Streptococcus pneumoniae isolated from patients who were treated for pneumonia with levofloxacin contained mutations in both parC (DNA topoisomerase IV) and gyrA (DNA gyrase), known to confer fluoroquinolone resistance (see, Urban C, et al., J Infect Dis. 2001 September 15; 184(6):794-8; Schmitz F J, et al., Antimicrob Agents Chemother. November 2001;44(11):3229-31; Ince D, et al., Antimicrob Agents Chemother. December 2000;44(12):3344-50; Pan X S, et al., Antimicrob Agents Chemother. November 2001;45(11):3140-7; Richardson D C, et al., Antimicrob Agents Chemother. June 2001;45(6):1911-4; Roychoudhury S, et al., Antimicrob Agents Chemother. April 2001;45(4):1115-20; and Barnard F M, et al., Antimicrob Agents Chemother. July 2001;45(7):1994-2000), hereby incorporated by reference.

Amplification of the gyrA Gene

Fluroquinolone resistant Streptococcus pneumoniae was isolated from lung cultures of patients diagnosed with bronchial pneumonia. The bacterial nucleic acid was extracted from the samples by alkaline lysis. Primers for PCR designed to amplify the gyrA gene and the amplification conditions are set forth in Pan et al., and Barnard et al., supra.

Expression of the Protein

The PCR-generated DNA templates were directly transcribed and translated in vitro using a coupled reticulocyte lysate system, TNT T7 Quick for PCR DNA (Promega, Madison, Wis.). A 100 kDa protein, corresponding to the DNA gyrase A protein, was produced from this eukaryotic expression system. The protein was purified according to the method of Brown PO, et al., Proc Natl Acad Sci USA December 1979;76(12):6110-9. The size and integrity of the protein was confirmed by Western Blot.

Functional Assay for the Protein

The functional activity of the purified mutant DNA gyrase A protein obtained from the fluoroquinolone resistant Streptococcus pneumoniae was compared to wild-type DNA gyrase A protein in supercoiling inhibition assays and DNA cleavage assays as described in Pan et al., and Barnard et al., supra. A concentration range of antibiotics was added to contact both the wild-type and mutant proteins. In addition, the affinities for each antibiotic and both the wild-type and mutant proteins were derived according to the method described in Roychoudhury, et al., supra. In each assay, the following antibiotics were tested: ciprofloxacin, gatifloxacin, grepafloxacin, levofloxacin, trovafloxacin, gemifloxacin, monifloxacin, sparfloxacin, rifampin, muprocin, premafloxacin, and several 8-methoxy-nonfluorinated quinolones (NFQ's).

Interpretation of Phenotype: Drug Susceptibility

In enzyme inhibition or DNA cleavage assays, the mutant enzyme demonstrated an increase in the MIC (minimum inhibitory concentration) required to inhibit activity compared to wild-type of about 4-fold with sparfloxacin, about 50-fold with ciprofloxacin, and 32-fold with premafloxacin. The MICs for ciprofloxacin, gatifloxacin, grepafloxacin, levofloxacin, and trovafloxacin were above the maximal serum drug concentrations reported for standard dosage regimens. In contrast, the MICs for the NFQs, clinafloxacin, gemifloxacin and moxifloxacin were below the maximal serum concentrations. Clinically, this would suggest discontinuing ciprofloxacin, gatifloxacin, grepafloxacin, levofloxacin, and trovafloxacin, continuing therapy with the NFQ's, clinafloxin, gemifloxacin and moxifloxacin, and monitoring for further changes in activity for sparfloxacin and premafloxacin.

In binding assays, the NFQs and clinafloxacin showed higher affinities toward both the wild-type and mutant DNA Gyrase A targets than ciprofloxacin, trovafloxacin, gatifloxacin, gemifloxacin and moxifloxacin. Furthermore, the ratio of the calculated affinity parameter for DNA gyrase to that for a control protein, topoisomerase IV, was lower in the case of the NFQs, clinafloxacin, and gatifloxacin than in the case of ciprofloxacin and trovafloxacin. Taken in combination, the results from both experiments suggest to one skilled in the art that the NFQ and clinafloxacin quinolones are better able to exploit multiple drug targets, resistance has not yet developed in the target protein, and that anti-bacterial inhibition can be achieved within pharmacologically acceptable dose ranges. Thus a physician or clinician is able to elect a course of chemotherapy against the fluoroquinolone resistant Streptococcus pneumoniae, that has the highest probability of ameliorating the disease state.

### EXAMPLE 7

Anti-Fungal Resistance.

Most anti-fungal drugs possess mechanisms of action aimed at disrupting the integrity of the fungal cell membrane by either interfering with the biosynthesis of membrane sterols or by inhibiting sterol functions. However, one significant obstacle preventing successful anti-fungal therapy is the dramatic increase in drug resistance, especially against azole antimycotics. Among the major mechanisms by which fungi invoke drug resistance is the overexpession of extrusion pumps able to facilitate the efflux of cytotoxic drugs from the cell thus leading to decreased drug accumulation and diminished concentrations. Since the initial observations that azole resistance by fungi may be caused by overexpression of multidrug efflux transporter genes, significant advances have been achieved primarily with Saccharomyces cerevisiae, Candida albicans, Aspergillus and Cryptococcus. Analysis of the transport functions of individual Candida albicans plasma membrane drug efflux pumps is hampered by the multitude of endogenous transporters. The protein Cdr1p is the major pump implicated in multiple-drug-resistance phenotypes, and can be amplified from the genomic PDR5 locus in a Saccharomyces cerevisiae mutant (AD 1-8u(−)) from which seven major transporters of the ATP-binding cassette (ABC) family have been deleted. S. cerevisiae AD1-8u(−) demonstrates a drug sensitive phenotype and is hypersensitive to azole antifungals (the MICs at which 80% of cells were inhibited [MIC(80)s] at such typical drug doses are 0.625 g/ml for fluconazole, <0.016 g/ml for ketoconazole, and <0.016 g/ml for itraconazole), whereas, for example a strain (AD 1002) that overexpresses C. albicans Cdr1p was resistant to azoles [(MIC(80)s] of fluconazole, ketoconazole, and itraconazole, 30, 0.5, and 4 & g/ml, respectively). See, Nakamura K, Antimicrob Agents Chemother. December 2001;45(12):3366-3374, incorporated herein by reference. Other such targets for bioactive molecules that are correlated with the resistant mechanisms of Candida albicans to fluconazole (FCZ) include the 14-alpha-demethylase gene (ERG16 gene), the lanosterol 14alpha-demethylase gene (ERG11) and the genes encoding the efflux transporters (MDR1 and CDR), and the cyp51-related genes (cyp51A and cyp51B) encoding 14-alpha sterol demethylase-like enzymes identified in the opportunistic human pathogen Aspergillus fumigatus. Amplification conditions and primer sets are disclosed in Wang W, et al., Chin Med J (Engl). May 1999;112(5):466-71, Perea S, et al., Antimicrob Agents Chemother. October 2001;45(10):2676-84, and Mellado E, et al., J Clin Microbiol. July 2001;39(7):2431-8, see also, St. Georgiev V., Curr Drug Targets. November 2001;1(3):261-84, incorporated herein by reference.

Amplification of the gyrA Gene

C. albicans strains displaying high-level fluconazole resistance (MICs, μg/ml) were isolated from human immunodeficiency virus (HIV)-infected patients with oropharyngeal candidiasis. The levels of expression of genes encoding lanosterol 14alpha-demethylase (ERG11) and efflux transporters (MDR1 and CDR) implicated in azole resistance were monitored in matched sets of susceptible and resistant isolates. In addition, ERG11 genes were amplified by PCR as described in Perea S, et al., Antimicrob Agents Chemother. October 2001;45(10):2676-84, incorporated herein by reference.

Expression of the Protein

The PCR-generated DNA templates were directly transcribed and translated in a cell-free expression system using a coupled reticulocyte lysate system, TNT T7 Quick for PCR DNA (Promega, Madison, Wis.). A 60 kDa protein, corresponding to the lanosterol 14 alpha-demethylase protein, was produced from this eukaryotic expression system. The protein was purified according to the method of Kalb VF, et al., DNA, Dec;6(6):529-37 (1987), incorporated herein by reference. The integrity and size of the purified protein was confirmed by Western Blot.

Functional Assay for the Protein

Microsomes were isolated from C. albicans as described in Marichal, P., et al., Microbiology (1999), 145, 2701-2713, incorporated herein by reference. Prevention of CO-complex formation in the reduced microsomal cytochrome P450 preparation provides an assay that can be used to test the affinity of the protein for an azole. The P-450 content and the effects of azoles on the interaction of CO with the reduced haem iron of P-450 were measured as described in Vanden Bossche, H., et al., Drug Dev Res, 8:287-298 (1986), incorporated herein by reference. The assay employed 0.1 nmol cytochrome P450 and 100 pM to 100 μM ranges of the anti-fungal compounds itraconazole and fluconazole.

Interpretation of Phenotype: Drug Susceptibility

IC₅₀ values for itraconazole for the wild-type lanosterol 14 alpha-demethylase protein is typically reported in the 10-50 nM range. IC₅₀ values for the mutant proteins ranged from 30-75 nM, while at 100 nM, the drug caused a near complete inhibition of the mutant lanosterol 14 alpha-demethylase proteins. The mutant strains can be regarded as itraconazole-sensitive. For fluconazole, more pronounced differences were observed. IC₅₀ values ranged more than 100-fold, from 40 nM for the wild-type proteins to about 4880 nM for the mutant proteins. The results suggest to one skilled in the art that a significant resistance to itraconazole has not yet developed in the mutant lanosterol 14 alpha-demethylase proteins, and that anti-fungal inhibition can still be achieved within pharmacologically acceptable dose ranges. Thus a physician or clinician is able to elect a course of chemotherapy against these C. albicans strains displaying high-level fluconazole resistance that has the highest probability of ameliorating the oropharyngeal candidiasis.

### EXAMPLE 8

The α4 Subunit of the VLA-4 Receptor

Inflammation is a response of vascularized tissues to infection or injury and is effected by adhesion of leukocytes to the endothelial cells of blood vessels and their infiltration into the surrounding tissues. In normal inflammation, the infiltrating leukocytes release toxic mediators to kill invading organisms, phagocytize debris and dead cells, and play a role in tissue repair and the immune response. However, in pathologic inflammation, infiltrating leukocytes are over-responsive and can cause serious or fatal damage. See, e.g., Hickey, Psychoneuroimmunology II (Academic Press 1990 incorporated by reference).

The attachment of leukocytes to endothelial cells is effected via specific interaction of cell-surface ligands and receptors on endothelial cells and leukocytes (see, Springer, Nature 346:425-433 (1990) incorporated by reference). The identity of the ligands and receptors varies for different cell subtypes, anatomical locations and inflammatory stimuli. The VLA-4 leukocyte cell-surface receptor was first identified by Hemler, EP 330,506 (1989) (incorporated by reference). VLA-4 is a member of the β1 integrin family of cell surface receptors, each of which comprises α and β chains. VLA-4 contains an α4 chain and a α1 chain. VLA-4 specifically binds to an endothelial cell ligand termed VCAM-1 (see, Elices et al., Cell 60:577-584 (1990) incorporated by reference). Although VCAM-1 was first detected on activated human umbilical vein cells, this ligand has also been detected on brain endothelial cells. See commonly owned, co-pending application U.S. Ser. No. 07/871,223 (incorporated by reference).

Adhesion molecules such as VLA-4, are potential targets for anti-autoimmune compounds, such as peptides and non-peptide compounds, biarylalkanoic acids, 4-amino-phenylalanine compounds, thioamide derivatives, cycli amino acid derivatives, and heterocyclic compounds, see U.S. Pat. Nos.: 6,306,887, 6,291,511, 6,291,453, 6,288,267, 5,998,447, and 6,001,809, the entirety of these patents are hereby incorporated by reference. The VLA-4 receptor is a particularly important target because of its interaction with a ligand residing on brain endothelial cells. Diseases and conditions resulting from brain inflammation have particularly severe consequences. For example, one such disease, multiple sclerosis (MS), has a chronic course (with or without exacerbations and remissions) leading to severe disability and death. The disease affects an estimated 250,000 to 350,000 people in the United States alone.

Antibodies against the VLA-4 receptor have been tested for their anti-inflammatory potential both in vitro and in vivo in animal models. See U.S. Ser. No. 07/871,223 and Yednock et al., Nature 356:63-66 (1992) incorporated by reference). The in vitro experiments demonstrate that anti-VLA-4 antibodies block attachment of lymphocytes to brain endothelial cells. The animal experiments test the effect of anti-VLA-4 antibodies on animals having an artificially induced condition (experimental autoimmune encephalomyelitis), simulating multiple sclerosis. The experiments show that administration of anti-VLA-4 antibodies prevents inflammation of the brain and subsequent paralysis in the animals. Collectively, these experiments identify anti-VLA-4 antibodies as potentially useful therapeutic compounds for treating multiple sclerosis and other inflammatory diseases and disorders (see U.S. Pat. No. 5,840,299, incorporated herein by reference).

The invention provides assays for expressing the α4 subunit of the VLA-4 receptor to assay for a MAb 21.6 binding phenotype. The binding phenotype determines the potential for methods of treatment that exploit the capacity of humanized MAb 21.6 to block α4-dependent interactions of the VLA-4 receptor. The α4-dependent interaction of the VLA-4 receptor with the VCAM-1 ligand on endothelial cells is an early event in many inflammatory responses, particularly those of the central nervous system. Undesired diseases and conditions resulting from inflammation of the central nervous system having acute clinical exacerbations include multiple sclerosis (Yednock et al., Nature 356, 63 (1992); Baron et al., J. Exp. Med. 177, 57 (1993)), meningitis, encephalitis, stroke, other cerebral traumas, inflammatory bowel disease (Hamann et al., J. Immunol. 152, 3238 (1994), ulcerative colitis, Crohn's disease, rheumatoid arthritis (van Dinther-Janssen et al., J. Immunol. 147, 4207 (1991); van Dinther-Janssen et al., Annals Rheumatic Diseases 52, 672 (1993); Elices et al., J. Clin. Invest. 93, 405 (1994); Postigo et al., J. Clin. Invest. 89, 1445 (1992), asthma (Mulligan et al., J. Immunol. 150, 2407 (1993) and acute juvenile onset diabetes (Type 1) (Yang et al., PNAS 90, 10494 (1993); Burkly et al., Diabetes 43, 529 (1994); Baron et al., J. Clin. Invest. 93, 1700 (1994). The entirety of these papers are hereby incorporated by reference.

Amplification of the VLA-4 Gene Fragment.

The sequence of the VLA-4 receptor is set forth in Genbank, sequence acsession numbers NM/000885 and XM/002572. The nucleotide sequence encoding the α4 subunit is amplified or otherwise provided by the methods described herein.

Expression of the Protein

The expression of the α4 subunit of the VLA-4 receptor is expressed by the methods descibed herein. The integrity of the protein is confirmed by Western blot using the monoclonal antibody MAb 21.6 from ascites at a 1:50 dilution. The antibody recognizes the native (functional) protein subunit, thus providing another way of detecting a binding phenotype.

Functional Assay for the Protein

Humanized monoclonal antibodies to the α4 subunit of the VLA-4 receptor are described in U.S. Pat. No. 5,840,299 as described. The monoclonal antibody MAb 21.6 was used for surface plasmon resonance assays to measure affinity of the mAb for the α4 subunit of the VLA-4 receptor. Ten nmols of the purified α4 subunit of the VLA-4 receptor protein was affixed via succinimide ester coupling to a BIAcore® chip, and equilibrated as described in the BIAcore® users manual. The monoclonal antibody MAb 21.6 was added to the flow cell in 10 pM, 25 pM, 50 pM, 75 pM, 100 pM, 250 pM, 500 pM, 750 pM, 1 nM, 2.5 nM, 5 nM, 7.5 nM, 10 nM, 25 nM, 50 nM, 100 nM, and 1000 nM concentrations. The association and dissociation constants for the reaction were used to calculate the binding constant (K_{D}) for the receptor subunit and MAb.

Interpretation of Phenotype: Drug Susceptibility

The K_{D} was determined to be approximately 10⁻⁹. This value suggests a moderately strong affinity for the target by the Mab 21.6. This indicates that anti-α4 subunit therapy with Mab 21.6 provides a method of inhibiting the VLA-4 receptor. MAb 21.6 was compared with another antibody against.α4 integrin called L25. L25 is commercially available from Becton Dickinson, and has been reported in the literature to be a good inhibitor of α4β1 integrin adhesive function. The capacity to block activated α4β1 integrin is likely to be of value in treating inflammatory diseases such as multiple sclerosis.

As a further comparison between MAb 21.6 and L25, the capacity of antibody to inhibit human T cell adhesion to increasing amounts of VCAM-1 was determined. In this experiment, increasing amounts of VCAM-1 were coated onto plastic wells of a 96 well assay plate, and the ability of the human T cell line, Jurkat (which expresses high levels of α4β1 integrin), to contact and bind to the coated wells was measured. The results indicate that L25 is a good inhibitor of cell adhesion when low levels of VCAM-1 are encountered, but becomes completely ineffective at higher levels of VCAM-1. MAb 21.6, on the other hand, inhibits cell adhesion completely, regardless of the amount of VCAM-1 present. The capacity to block at high concentrations of VCAM-1 is desirable for therapeutic applications because of upregulation of VCAM-1 at sites of inflammation (see, U.S. Pat. No. 5,840,299 incorporated herein by reference).

### EXAMPLE 9

Tyrosine Kinases

The present invention relates to compounds which inhibit tyrosine kinase enzymes, compositions which contain tyrosine kinase inhibiting compounds and methods of using tyrosine kinase inhibitors to treat tyrosine kinase-dependent diseases and conditions such as neoangiogenesis, cancer, tumor growth, atherosclerosis, age related macular degeneration, diabetic retinopathy, inflammatory diseases, and the like in mammals. The invention provides for an assay and method of expressing a tyrosine kinase or tyrosine phosphatase protein, to determine its phenotype.

Kinases regulate many different cell proliferation, differentiation, and signaling processes by adding phosphate groups to proteins. Uncontrolled signaling has been implicated in a variety of disease conditions including inflammation, cancer, arteriosclerosis, and psoriasis. Reversible protein phosphorylation is the main strategy for controlling activities of eukaryotic cells. It is estimated that more than 1000 of the 10,000 proteins active in a typical mammalian cell are phosphorylated. The high energy phosphate which drives activation is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc), cell cycle checkpoints, and environmental or nutritional stresses and is roughly analogous to turning on a molecular switch. When the switch goes on, the appropriate protein kinase activates a metabolic enzyme, regulatory protein, receptor, cytoskeletal protein, ion channel or pump, or transcription factor. Inhibitors of protein kinases include angiogenesis inhibitors, pyrazole derivatives, cyclin-C variants, aminothiazole compounds, quinazoline compounds, benzinidazole compounds, polypeptides and antibodies, pyramidine derivatives, substituted 2-anilopyramidines, and bicyclic heteroaromatic compounds (see, U.S. Pat. Nos. 6,265,403, 6,316,466, 6,306,648, 6,262,096, 6,313,129, 6,162,804, 6,096,308, 6,194,186, 6,235,741, 6,235,746, 6,207,669, and 6,043,045, the entirety of these patents are hereby incorporated by reference).

Protein tyrosine kinases, PTKs, specifically phosphorylate tyrosine residues on their target proteins and may be divided into transmembrane, receptor PTKs, and nontransmembrane, non-receptor PTKs. Transmembrane protein-tyrosine kinases are receptors for most growth factors. Binding of growth factor to the receptor activates the transfer of a phosphate group from ATP to selected tyrosine side chains of the receptor and other specific proteins. Growth factors (GF) associated with receptor PTKs include, for example: epidermal GF, platelet-derived GF, fibroblast GF, hepatocyte GF, insulin and insulin-like GFs, nerve GF, vascular endothelial GF, and macrophage colony stimulating factor.

Non-receptor PTKs lack transmembrane regions and, instead, form complexes with the intracellular regions of cell surface receptors. Some of the receptors that function through non-receptor PTKs include those for cytokines and hormones (growth hormone and prolactin) and antigen-specific receptors on the surface of T and B lymphocytes. The protein products of oncogenes and many growth-factor receptors have protein kinase activities that phosphorylate tyrosine.

Another family of kinases is the protein kinase C (PKC) family. Phosphorylation plays an essential role in regulating PKC. These enzymes transduce signals promoting phospholipid hydrolysis and are recruited to membranes upon the production of diacylglycerol and, for the conventional isoforms, increased Ca2+ concentrations. Binding of these cofactors results in conformational change that removes an autoinhibitory (pseudo substrate) domain from the active site, thus promoting substrate binding and phosphorylation. Apoptosis of prostate epithelial cells is regulated by activators and inhibitors of the PKC family. The PKC family of serine/threonine kinases has been associated with signal transduction regulation cell growth and differentiation but has recently been associated with the regulation of cell death (Day, M. L. et al., Cell Growth & Differ. 5: 735-741(1994); Powell, C. T. et al., Cell Growth & Differ. 7: 419-428(1996) incorporated herein by reference. Most PKC isozymes require the physiological activator diacylglycerol, which is derived from membrane phospholipids. Additionally, PKC activity also requires association with cellular membranes and/or cytoskeletal components to execute many of its physiological functions. PKC modulates signal transduction pathways that have been linked to both positive and negative regulation of the cell cycle and the initiation of apoptosis. An example of a PKC which is involved in the growth-inhibitory action of transforming growth factor-betal (TGF-β1) in PC3, a human prostate cancer cell line, is protein kinase K02B12 from C. elegans.

RNA-activated protein kinase (PKR) is a serine/threonine protein kinase induced by interferon treatment and activated by double stranded RNAs. When PKR becomes autophosphorylated, it catalyzes phosphorylation of the alpha subunit of protein synthesis eukaryotic initiation factor 2 (eLF-2). Protein kinase inhibitors (PKI) have demonstrated potential for their use in the treatment of human cancers, in particular leukemia. (Lock, R. B. Cancer Chemother. Pharmacol. 39(5): 399-409(1997), incorporated herein by reference. An example of a serine/threonine kinase inhibitor is the P58 PKR inhibitor (PKRI) from B. taurus, a 504-amino acid hydrophilic protein. PKRI, expressed as a histidine fusion protein in E. coli, blocked both the autophosphorylation of PKR and phosphorylation of the alpha subunit of eLF-2. Western blot analysis showed that PKRI is present not only in bovine cells but also in human, monkey, and mouse cells, suggesting the protein is highly conserved. Another example of an inhibitor of protein kinase C is the protein kinase inhibitor from mouse, which acts as an inhibitor of cAMP-dependent protein kinase and protein kinase C.

Thus, the discovery of a new PK's and PKI's and the polynucleotides encoding them satisfies a need in the art by providing new compositions which are useful in the diagnosis, prevention, and treatment of diseases associated with cell proliferation, and in particular, cancer, immune responses, and development disorders (see, U.S. Pat. No. 6,194,186, incorporated herein by reference.

Amplification and Expression of Bioactive Molecules

Protein kinases and protein phosphatases are selected depending on the experimental design or clinical determination. Amplification and expression is effectuated by the methods described.

Phenotypic Assays

Protein kinases and protein phosphatases are extensively studied molecules. Simple and efficient testing methods for determining kinase or phosphatase activity can be purchased from Promega, such as the SigmaTECT® Protein Kinase Assay, and the Non-Radioactive Phosphatase Assay System. Numerous peptide substrates for measuring kinase activity are also described in the scientific literature, such as Kemp, BE, et al., J Biol Chem 252, 4888 (1977); Casinelle, J E, et al., Meth. Enzymol., 200 115 (1991) incorporated herein by reference. Pure preparations of enzymes and inhibitors are commercially available from a wide number of sources. These assays provide methods for determining the phenotype of the protein kinase and protein phosphatase. Phenotypic information is thus used in the drug discovery process to find compounds that can modulate the phenotype of these proteins.

### EXAMPLE 10

P-Glycoprotein

Multiple Drug Resistance in Cells

Certain cells are capable of developing resistance to drugs. Hamster, mouse and human tumor cell lines displaying multiple-drug resistance (MDR) have been reported. A major problem in the chemotherapy of cancer is the development of cross-resistance of some human tumors to multiple chemotherapeutic drugs. The type of multiple-drug resistance is accompanied by a decrease in drug accumulation and an increase in the expression of a multiple drug resistance protein, which is also known as P-glycoprotein or gp 170. (The term “P-glycoprotein” shall denote both P-glycoprotein and gp 170). P-glycoprotein is a high molecular weight membrane protein (Mw 170-180 kDa) encoded by the MDR1 gene which is often amplified in MDR cells. The complete nucleotide sequence of the coding region of the human MDR1 gene and the complete corresponding amino acid sequence are disclosed in Patent Cooperation Treaty patent application, publication number WO 87/05943, priority date Mar. 28, and Aug. 1, 1986, “Compositions and methods for clones containing DNA sequences associated with multi-drug resistance in human cells,” to Roninson, I. B. A method of isolating cDNA specific for P-glycoprotein is described in European Patent Application, Publication No. 174,810, date of publication, Mar. 3, 1986, incorporated herein by reference,

While the “classical” MDR phenotype is based on P-glycoprotein, the “non-classical” MDR phenotype is based on other mechanisms, some of them as yet undefined. Tthe term “MDR phenotype” shall include both the classical and non-classical MDR phenotypes. “MDR markers” or “MDR antigens” include P-glycoprotein and other antigens expressed solely or differentially on cells expressing the MDR phenotype. Different mutant cell lines exhibit different degrees of drug resistance. Examples of cell lines exhibiting the MDR phenotype have been selected for resistance to a single cytotoxic compound. These cell lines also display a broad, unpredictable cross-resistance to a wide variety of unrelated cytotoxic drugs having different chemical structures and targets of action, many of which are used in cancer treatment. This resistance impedes the efficacy of drugs used in chemotherapy to slow down or decrease the multiplication of cancerous cells.

A monoclonal antibody that is capable of recognizing the K562/ADM adriamycin-resistant strain of a human myelogenous leukemia cell line K562 has been disclosed in European Patent Application, Publication No. 214,640 A3, “Monoclonal antibody in relation to drug-resistant cancers and productions thereof,” to Tsuruo, T., published Mar. 18, 1987, incorporated by reference. This monoclonal antibody is produced by a hybridoma formed as a fusion product between a mouse myeloma cell and a spleen cell from a mouse that has been immunized with the K562/ADM strain.

Fc Receptors (FcRs)

Fc receptors are found on many cells which participate in immune responses. Fc receptors (FcRs) are cell surface receptors for the Fc portion of immunoglobulin molecules (Ig). Among the human Ig's that have been identified so far as able to bind Fc receptors are IgG (FcRn, FcγRI, FcγRII, and FcγRIII), IgE (FcεR), IgA (FcαR), and polymerized IgM/A (FcμαR). The different kinds of FcRs are found in the following cell types, for example, mast cells, macrophages, monocytes, eosinophils, platelets, leukocytes, neutrophils, glandular epithelium, hepatocytes, kidney, heart, placenta, lung, and pancreas, see, Hogg, N., Immun. Today, 9:185-86 (1988); Unkeless, J. C., Ann. Rev. 1 mm., 6:251-87 (1992), Burmeister etal., Nature. November 24;372(6504):336-43 (1994), and Simister N E., Vaccine Aug-Sep;16(14-15):1451-5 (1998), incorporated herein by reference. Structure and function of FcR's provide a crucial link between effector cells and the lymphocytes that secrete Ig, as well as IgG homeostasis.

Hybridoma 3G8 is a murine hybridoma which secretes a mouse IgG1 MAB that recognizes human FcγRIII on human and chimpanzee leukocytes. For example, MAB 3G8 recognizes FcγRIII on neutrophils, monocytes, macrophages, and NK cells. MAb and hybridoma 3G8 are described in Unkeless, et al., supra, and was initially disclosed in Unkeless, J. C., etal., J. Exp. Med, 150:580-596 (1979) incorporated herein by reference.

A chemically constructed bispecific antibody consisting of MAB 3G8 chemically cross-linked to a melanoma specific MAB could direct Fc.gamma.RIII bearing lymphocytes to kill melanoma cells both in vitro and in nude mice. Titus, J. A., et al., J. Immunol., 139:3153 (1987) incorporated by reference. Further, another chemically constructed bispecific antibody anti-CD3/MRK16, was reactive with P-glycoprotein on MDR cells and CD3 antigen on T-lymphocytes. The anti-CD3/MRK16 bispecific antibody was found to induce lysis of MDR tumor cells in vitro. Van Dijk, J. et al., Int. J. Cancer, 44: 738 (1989) incorporated by reference. Other inhibitors of P-glycoprotein include the monoclonal antibodies described in U.S. Pat. Nos. 6,143,837, and 6,106,833, kinase C inhibitors, anthranilic acid derivatives, oligonucleotides, thrphenylpiperidine compounds, tetraarylethylene compounds, and diarylalkyl compounds. These inhibitor compounds are described in U.S. Pat. Nos. 5,972,598, 6,218,393, 6,001,991, 5,670,521, 5,665,780, 5,648,365, 6,043,045, and 5,837,536, hereby incorporated by reference.

The P-glycoprotein sequence can be found at Genbank No.:M14758. The functioanl protein can be expressed using the methods described herein. The affinity for anti-P-glycoprotein Mab's can be determined by ELISA binding assay, or SPR. Structural changes to the protein in the presence or absence of the inhibitory compound can be detected through mass spectroscopy and by 2-D NMR. The detection of a drug resistant phenotype is suggestive of potential therapies. The performance of such assays to determine the phenotype, and the interpretation of such phenotype re know to medical professionals and those similarly skilled in the art.

### Equivalents

From the foregoing detailed description of the specific embodiments of the invention, it should be apparent that a unique procedure to express and assay a biomolecule for a clinically relevant phenotype has been described resulting in improved patient therapies and the drug discovery process. Although particular embodiments have been disclosed herein in detail, this has been done by way of example for purposes of illustration only, and is not intended to be limiting with respect to the scope of the appended claims which follows. In particular, it is contemplated by the inventor that substitutions, alterations, and modifications may be made to the invention without departing from the spirit and scope of the invention as defined by the claims. For instance, the choice of bioactive molecule for assay, or the choice of chemotherapeutic agent, or the choice of appropriate patient therapy based on the assay is believed to be matter of routine for a person of ordinary skill in the art with knowledge of the embodiments described herein.

We claim:

1. A method for producing and evaluating a bioactive molecule comprising the steps of:
a) providing a nucleic acid sequence comprising a bioactive molecule;
b) expressing the bioactive molecule encoded by the nucleic acid sequence obtained in step (a), wherein the expressed bioactive molecule has a detectable phenotype;
c) contacting the bioactive molecule obtained in step (b) with a compound; and
d) detecting the phenotype of the bioactive molecule in the presence or absence of the compound contacted in step (c).

2. The method of claim 1, wherein the bioactive molecule is selected from the group consisting of: a viral molecule, a bacterial molecule, a fungal molecule, a protozoal molecule, a human molecule and an animal molecule.

3. The method of claim 1, wherein the bioactive molecule is a protein further comprising a retrovirus protein, a herpesvirus protein, a hantavirus protein, a hepatitis virus protein, an influenza protein, a myxovirus protein, a picomavirus protein, an adenovirus protein, a poxvirus protein, a flavivirus protein or a coronavirus protein.

4. The method of claim 1, wherein the bioactive molecule is a protein further comprising a streptococcus protein, a staphylococcus protein, an enterococus protein, a neisseria protein, a salmonella protein, a mycubacteria protein, a bacillus protein, a mycoplasma protein, a chlamydia protein, a francisella protein, a pasturella protein, a brucella protein, a pseudomonas protein, a listeria protein, a clostridium protein, a yersinia protein, a vibrio protein, a shigella protein, or an enterobacteriaceae protein.

5. The method of claim 1, wherein the bioactive molecule is a protein further comprising a plasmodium protein, a trypanosome protein, or a crytosporydium protein.

6. The method of claim 1, wherein the bioactive molecule is a protein further comprising a candida protein, a cryptococcus protein, a malassezia protein, a histoplasma protein, a coccidioides protein, a hyphomyces protein, a blastomyces protein, an asp ergillus protein, a penicillium protein, a pseudallescheria protein, a fusarium protein, a paecilomyces protein, a mucor/rhizopus protein, a pneumocystis protein, a rhinosporidium protein, a sporothrix protein, a trichophyton protein, a microsporum protein, a epidermophyton protein, a basidiobolus protein, a conidiobolus protein, a rhizopus protein, a cunninghamelia protein, a paracoccidioides protein, a pseudallescheria protein, or a rhinosporidium protein.

7. The method of claim 1, wherein the nucleic acid sequence encoding the biomolecule further comprises deoxyribonucleic acid or ribonucleic acid.

8. The method of claim 1 or claim 7, wherein the nucleic acid sequence encoding a bioactive molecule further comprises transfer RNA or polyA+ RNA.

9. The method of claim 1, wherein the bioactive molecule further comprises a protein, a glycoprotein, a polysaccharide, a mucopolysaccharide, a lipopolysaccharide, a lipoprotein, a carbohydrate, or a nucleic acid.

10. The method of claim 1, wherein the bioactive molecule encoded by the nucleic acid is expressed in a cell-free eukaryotic cell lysate translation system.

11. The method of claim 1, wherein the bioactive molecule encoded by the nucleic acid is expressed in a cell-free prokaryotic cell lysate translation system.

12. The method of claim 10, wherein the bioactive molecule encoded by the amplified nucleic acid sequence is expressed in a cell-free reticulocyte lysate translation system.

13. The method of claim 12, wherein the bioactive molecule encoded by the amplified nucleic acid sequence is expressed in a cell-free reticulocyte lysate coupled transcription/translation system.

14. The method of claim 13, wherein the bioactive molecule encoded by the nucleic acid sequence and expressed in a cell-free reticulocyte lysate coupled transcription/translation system is a nucleic acid selected from the group consisting of: deoxyribonucleic acid, ribonucleic acid, polyA+ RNA, tRNA, and rRNA.

15. The method of claim 1, wherein the nucleic acid sequence that encodes the bioactive molecule further comprises a second nucleic acid sequence operably linked to said bioactive molecule.

16. The method of claim 15, wherein the second nucleic acid sequence comprises a regulatory element.

17. The method of claim 15, wherein the second nucleic acid sequence comprises a purification motif.

18. The method of claim 15, wherein the second nucleic acid sequence encodes a gene product or fragment thereof comprising a purification motif.

19. The method of claim 1, wherein the bioactive molecule is contacted with a compound selected from the group consisting of: an anti-viral compound, an anti-bacterial compound, an anti-fungal compound, an anti-cancer compound, an immunosuppressive compound, a hormone, a cytokine, a lymphokine, a chemokine, an enzyme, a polypeptide, a polynucleotide, and a nucleoside analogue.

20. The method of claim 1, wherein detecting the phenotype of the bioactive molecule further comprises assaying the enzymatic activity of the bioactive molecule.

21. The method of claim 20, wherein assaying the enzymatic activity of the bioactive molecule further comprises assaying the bio active molecule for a resistance phenotype to the compound.

22. The method of claim 1, wherein detecting the phenotype of the bioactive molecule further comprises assaying the affinity of the bioactive molecule for the compound.

23. The method of claim 22, wherein assaying the affinity of the bioactive molecule for the compound further comprises assaying the bioactive molecule for a resistance phenotype to the compound.

24. The method of claim 1, wherein detecting the phenotype of the bioactive molecule further comprises assaying the structure of the bioactive molecule.

25. The method of claim 24, wherein assaying the structure of the bioactive molecule comprises predicting a resistance phenotype to the compound.

26. The method of claim 1, wherein the method is preceeded by the step of: amplifying a nucleic acid sequence in a cell-free system, wherein the nucleic acid sequence comprises a bioactive molecule.

27. The method of claim 1, wherein the nucleic acid encoding a bioactive molecule is amplified by a reaction selected from the group consisting of: a polymerase chain reaction, a ligase chain reaction, a transcription mediated amplification reaction, a nucleic acid sequence based amplification reaction, and a strand displacement amplification reaction.

28. The method of claim 1, wherein amplifying the nucleic acid encoding the biomolecule comprises a polymerase chain reaction further comprising one or more nested primer sets.

29. The method of claim 1, wherein amplifying the nucleic acid encoding the biomolecule NO:2, SEQ ID NO:3, SEQ ID NO:4.

30. The method of claim 1 or claim 26, wherein the method is preceeded by the step of: extracting one or more specemins from a patient afflicted with a disease state, wherein the specemins comprise a bioactive molecule associated with a disease state.

31. A method for producing and evaluating a bioactive molecule comprising the steps of:
a) isolating at least one organism or tissue, wherein the organism or tissue comprises a bioactive molecule associated with a disease state;
b) amplifying a nucleic acid sequence in a cell-free system, wherein the nucleic acid sequence comprises the bioactive molecule and is obtained from the organism or tissue isolated in step (a);
c) expressing the bioactive molecule encoded by the nucleic acid sequence obtained in step (b), wherein the expressed bioactive molecule has a detectable phenotype further comprising resistance to a first compound;
d) contacting the bioactive molecule obtained in step (c) with a second compound; and
e) detecting the phenotype of the bioactive molecule in the presence or absence of the second compound contacted in step (d).

32. The method of claim 1, wherein the method is preceeded by the step of: amplifying a nucleic acid sequence in a cell-free system, wherein the nucleic acid sequence comprises a bioactive molecule.

33. The method of claim 1 or claim 26, wherein the method is preceeded by the step of: extracting one or more specemins from a patient afflicted with a disease state, wherein the specemins comprise a bioactive molecule associated with the disease state.

34. A method for producing and evaluating a bioactive molecule comprising the steps of:
a) isolating at least one organism or tissue, wherein the organism or tissue comprises a bioactive molecule associated with a disease state;
b) amplifying a nucleic acid sequence in a cell-free system, wherein the nucleic acid sequence comprises the bioactive molecule and is obtained from the organism or tissue isolated in step (a);
c) expressing the bioactive molecule encoded by the nucleic acid sequence obtained in step (b), wherein the expressed bioactive molecule has a detectable phenotype further comprising resistance to a first compound;
d) contacting the bioactive molecule obtained in step (c) with a second compound; and
e) detecting the phenotype of the bioactive molecule in the presence or absence of the second compound contacted in step (d).
